(19) United States
(12) Patent Application Publication
(10) Pub. No.: US 2004/0133944 A1
(43) Pub. Date: Jul. 8, 2004

(54) Seed oil suppression to enhance yield of commercially important macromolecules

(75) Inventors: Kater Davis Hake, Cleveland, MS (US); Thomas Arthur Kerby, Greenville, MS (US); Harry Benjamin Collins, Scott, MS (US); Don Lee Keim, Leland, MS (US)

Correspondence Address: ROTHWELL, FIGG, ERNST & MANBECK, P.C., 1425 K STREET, N.W., SUITE 800, WASHINGTON, DC, 20005, US

(73) Assignee: Delta and Pine Land Company, Scott, MS

(21) Appl. No.: 10/647,140

(22) Filed: Aug. 25, 2003

Related U.S. Application Data
(60) Provisional application No. 60/438,500, filed on Jan. 8, 2003.

(51) Int. Cl. A01H 1/00; C12N 15/82

(52) U.S. Cl. 800/281

(57) ABSTRACT

This invention relates to a method for making a genetically modified cotton plant by regenerating a whole plant from a plant cell that has been transfected with DNA sequences including a gene, the expression of which results in suppression of oil biosynthesis in the developing seed. Plants made according to this method exhibit increased production of fiber. Also disclosed is a method for making a non-genetically modified cotton plant with reduced seed-oil content by selecting native alleles or alleles produced through mutagenesis that result in reduced oil content with resulting enhanced fiber yield. Methods are disclosed for developing commercially acceptable cultivars that contain the cottonseed-oil suppression trait. Plant cells, plant tissues, plant seed and whole plants containing the above DNA sequences and alleles form part of the invention.

This application claims the benefit of co-pending U.S. provisional application Serial No. 60/438,500, filed Jan. 8, 2003.

### BACKGROUND

1. Technical Field

This invention relates to methods for developing commercially acceptable cultivars and hybrids in cotton (e.g., Gossypium hirsutum L., G. barbadense L., G. arboreum and G. herbaceum) and other plants by suppression of the lipid biosynthetic pathway that impacts oil content, or oil storage mechanisms, resulting in enhanced yield of carbohydrate or protein products, for example fiber. More specifically, the invention relates to the identification, production and selection of plants that express or contain natural or modified seed-oil suppression genes, including alleles of genes, that affect seed-oil content. In some embodiments, the invention involves generating transgenic plants that contain within their genomes genetic systems that use cosuppression (or gene silencing), antisense, immunomodulation, ribozyme, transcription factor suppression or RNA interference (RNAi) strategies to suppress seed-oil biosynthesis pathways or storage mechanisms. The invention also provides a plant breeding method suitable for producing plants expressing a yield enhancing trait.

2. Description of the Background Art

Yield enhancement of commercially valuable products using traditional plant breeding techniques has been a main objective of both early and modern plant breeding efforts; steady and substantial gains have been achieved. Transgenic technology provides new opportunities to accelerate the genetic yield gain beyond that possible using these traditional methods by altering the metabolic machinery of the plant (see Dunwell, 2000 and Richards, 2000 for recent reviews). Efforts to enhance crop yield with transgenic technology have included the following strategies: improved photosynthetic efficiency (Barry et al., 2002; Ishimura et al., 1998; Ku et al., 1999; Miyagawa et al., 2001; Osteryoung, 1999; Xue et al., 2002; Sonnewald et al., 2001), carbohydrate metabolism (Barry et al., 2001; Haigler et al., 2001; Tomes et al., 2000; Quanz, 2000; Staub et al., 2000; Kossmann et al., 1997; Barry et al., 1998; Van Assche et al., 1999; Shoseyov et al., 2001; Barry et al., 2002; Ellis et al., 1998; Kerr, 1993; Regierer et al., 2002; Smeekens et al., 1999; Smeekens et al., 2000; Sun et al., 2001; Smidansky et al., 2002a; Smidansky et al., 2002b; Sonnewald et al., 1996; Willmitzer et al., 2001; Willmitzer et al., 1999), hormone manipulation (Chory et al., 2001; Habben et al., 2000; Chory et al., 1998; Chory et al., 2000; Chory et al., 2002; Chory et al., 1997; Eriksson et al., 2001; Schmulling et al., 2001), stress and disease tolerance (Broadway et al., 2001; Gaxiola, 2002; Zhang et al., 2001), nitrogen metabolism (Lightfoot et al., 1999; Coruzzi et al., 2000; Coruzzi et al., 1999; Kisaka, 2002, Smith, 2002), cell cycle regulation (Doerner et al., 2001; Roberts et al., 2000; De Veylder et al., 2000; Doerner et al., 1998; De Veylder et al., 2002; Doerner et al., 2000; Inze et al., 2002; Miskolczi et al., 2001; Sun et a., 2001; Kim, 2002), signal transduction (Sheen et al., 2000; Zhong et al., 2000;), morphological manipulation (Martienssen et al., 2001; Jackson et al., 2001; Liljegren et al., 2002), and other strategies (Bailey et al., 1998; Bailey et al., 1999; Donn, 1998; Khush, 1999; Neuhaus et al., 1999; Smith et al., 1999)

Traditional methods of plant breeding also have yielded modest gains in increasing the oil and protein content of cotton seed, while decreasing the gossypol content (Bassett et al., 1996). Transgenic technology has been used to modify seed constituents, focusing on lipid or protein profile and increasing the sugar, oil or protein content. Willmitzer et al. (2000) have reported antisense suppression of starch and protein to augment sugar or protein content, while Lassner et al. (2002a, 2002b, 2002c) have suggested suppression of the lipid triacyglycerol in corn and soybeans to produce novel lipids. Lipid modification in oil seed crops (e.g., canola, rapeseed, sunflower, soybean, safflower and cotton) has been an active area of research focused on increasing total lipid content and altering the lipid profile. See Chapman et al., 2001: Liu et al., 2002a; Katavic et al., 1995; Ohlrogge et al., 1997; Taylor et al., 2001; Zou et al., 1997; Brown et al., 2002. Of the oil seed crops, only in cotton is the seed-oil of relatively low economic value compared to another natural yield component (cellulose).

Cotton has been selected and cultivated by man primarily for its fiber, since the seed contains gossypol that limits its whole-seed feed use to ruminant animals only (Brubacker et al., 1999). Although the seed now is used in the production of oil, meal and dairy feed, its economic value to the modern farmer for these uses is low in comparison to the economic value of the fiber, which is used to produce yam, fabric and textile garments. Despite the greater mass per acre of seed as compared to fiber, the even greater economic value per pound of fiber as compared to seed has encouraged plant breeders to select cotton primarily for increased fiber yield and fiber quality, and only secondarily for increased seed yield and seed quality. As a result of this intense selection for genetic improvements in fiber yield, the genetic contribution to fiber yield and lint percent (ratio of fiber to fiber-plus-seed) has increased during the last 50 years while seed size has declined (Bassett et al., 1996). Public and private cotton breeding programs have included seed value (primarily oil content) as a breeding objective (Cherry et al., 1986). Despite this, advances in oil content have been limited due to the competing breeding objective of fiber yield. Attempts to increase both fiber and oil yield together have met with limited success (Dani et al., 1999; Bassett et al., 1996).

Cotton fibers are single epidermal cells that protrude from the seed coat. Fibers are composed of carbohydrates, proteins, lipids and minerals, although the predominant constituent is polymeric residues of glucose, (β-1,4) glucan chains referred to as cellulose. Cellulose in cotton fibers is synthesized during the 50 days after anthesis using phloem-translocated sucrose produced in photosynthetic or senescing plant tissues. In the biosynthesis of cellulose, sucrose is split into the 2 constituent carbohydrate residues, glucose and fructose. Fructose is interconverted to glucose. The glucose residues then are available for synthesis of cellulose in the epidermal cell walls (Delmar, 1999). This biosynthetic pathway is energetically conserved due to the limited degradation without oxidation of sucrose prior to synthesis of cellulose.

Cotton seeds typically are composed of 3.5% w/w starch (Pettigrew, 2001), 20% w/w oil and 20% w/w protein (Rayburn et al., 1989-2001). Stored seed-oil is predominately (97%) triacyglycerol (TAG) derived from linoleic (18:2), palmitic (16:0) and oleic (18:1) fatty acids (Tzen et al., 1993). Although within a boll peak cellulose deposition precedes peak oil accumulation by approximately 15 days, in the plant, oil and cellulose biosynthesis accumulate concomitantly (McD. Stewart, 1986). Seed TAG is synthesized following the glycolytic degradation of sucrose into acetyl Co-A that is then used as precursors for lipid biosynthesis. The TAG biosynthetic pathway from sucrose is energetically inefficient because sucrose must be oxidized and degraded into 2-carbon acetyl residues prior to reduction and condensation into 16- to 18-carbon fatty acids, and then incorporation into TAG.

Although agronomic practices to manage varieties that have the potential to generate excess vegetative growth have been published (Kerby, 1996), methods to breed new cultivars that incorporate reduced seed-oil traits have not. The predominant method to breed commercial transgenic cotton cultivars is the backcross method, whereby a transgene donor is crossed with an elite recurrent parent to generate an F1 hybrid, which is then repeatedly backcrossed to the recurrent parent. Backcrosses are repeated 2 to 6 times, depending on the breeding objectives and genetic relatedness of the gene donor and recurrent parents. Once the desirable alleles from the recurrent parent are fixed in the resulting hybrid, selfing and selection of a transgenic variety with essentially the same characteristics of the recurrent parent is identified at the F2 and F3 generations. The backcross method has been used on all elite transgenic cotton varieties for which Plant Variety Protection (PVP) certificates were issued in the U.S. before Dec. 31, 2002. A modified backcross method, whereby two lines carrying different transgenes, but derived from the same recurrent parent, are crossed, also has been employed.

The traditional method of developing novel non-transgenic cultivars is forward crossing. This breeding strategy relies on multiple or single crosses between diverse elite and non-elite cultivars, several generations of selfing to fix alleles in a homozygous state, and then subsequent selection of novel allele combinations. Forward crossing has rarely been employed in transgenic breeding of cotton. In the early stages of new transgene commercialization, the number and diversity of cultivars carrying a new transgene are limited, thus forward crossing combinations are limited. Maximum fiber yield from cottonseed oil suppression traits will require novel breeding methods to incorporate these traits into high yielding adapted commercial cultivars, since selection for yield without the trait may not assemble the best combinations of alleles for cultivars containing the cottonseed oil suppression trait.

### SUMMARY OF THE INVENTION

This invention relates to a method of selecting and/or generating oil suppression genes, or alleles of genes that affect seed-oil biosynthesis or deposition, and a method of producing plants, including transgenic plants, wherein the seed-oil content is reduced, thereby increasing the supply of sucrose for protein and carbohydrate (including cellulose and starch) production within the seed. In one embodiment, the genetic control of seed-oil content is achieved by selecting naturally occurring or artificially induced seed-oil suppression alleles of endogenous genes that control seed-oil content and introducing them into elite germplasm using plant breeding strategies. In a further embodiment, the control of seed-oil content is achieved by introducing a gene the action of which directly alters seed-oil content. In yet a further embodiment, the genetic control of seed-oil content is achieved through application of an external stimulus that activates expression of a seed-oil suppressing gene. In yet a further embodiment, it is achieved by application of an external stimulus that activates an introduced genetic system that results in the suppression of seed-oil biosynthesis and/or storage without the need for the continued presence of the external stimulus. In yet a further embodiment, control of seed-oil content is achieved through hybridization and in a sixth embodiment, it is achieved by the direct introduction of an activation gene into a plant containing an inactive seed-oil suppression gene.

An object of embodiments of the invention, therefore, is to produce suppression of oil in the seed of plants, reducing the energy-intensive incorporation of sucrose-derived carbon into stored oil, thereby increasing the supply of sucrose for sustained fiber and plant vegetative growth during plant growth. A preferred embodiment involves increasing the supply of sucrose to a cotton plant during the boll-filling phase of plant growth.

An additional object of embodiments of the invention is to provide a breeding method for producing plants that contain a yield enhancing trait, such as a seed-oil suppression transgene or any other transgene, and also contain a genetic and phenotypic background that optimizes the performance of the yield enhancing trait.

Therefore, in one embodiment, the invention provides a reduced seed-oil content plant cell that expresses a seed-oil suppressing gene under control of a plant-active promoter which exhibits a reduction in seed-oil and a concomitant increase in plant carbohydrate, protein or both and where the seed-oil suppressing gene is selected from the group consisting of a mutant allele of a gene naturally occurring in said plant and a transgene. Preferred plants for use in the invention are selected from the group consisting of cotton, corn, soybean, canola and wheat. The invention provides, in another embodiment, a reduced seed-oil content plant which comprises a cell as described above. In yet another embodiment, the invention provides a reduced seed-oil content plant as described above that has enhanced fiber yield. Such reduced seed-oil content plants may be an elite or primitive cultivar.

The seed-oil suppressing gene may be a mutant allele of a gene naturally occurring in the plant or may be introduced into the germplasm of an elite cultivar. In some embodiments, the seed-oil suppressing gene controls seed-oil content by suppressing seed-oil biosynthesis or by suppressing seed-oil storage. In some embodiments of the invention, the seed-oil suppressing gene is generated within the germplasm of the plant by random mutagenesis, such as by exposure to ethyl methanesulfonate. In some embodiments of the invention, the seed-oil suppressing gene is identified and isolated from a mutagenized seed stock. Expression of the seed-oil suppressing gene suppresses at least one biosynthetic step in oil biosynthesis in preferred embodiments of the invention.

In some embodiments of the invention, expression of the seed-oil suppressing gene suppresses carbonic anhydrase, ACCase, lysophosphatidic acid acyltransferase, diacylglycerol acyltransferase, oleosin or any combination thereof. Preferably, the seed-oil suppressing gene suppresses a gene early in the oil biosynthetic pathway and a gene late in the oil biosynthetic pathway, such as the CA gene and/or the ACCase gene (early) and the LPAT gene and/or the DGAT gene (late).

Preferably, the seed-oil suppressing gene is a transgene. This transgene suppresses at least one biosynthetic step in oil biosynthesis, such as carbonic anhydrase, ACCase, lysophosphatidic acid acyltransferase, diacylglycerol acyltransferase, oleosin or any combination thereof. In preferred embodiments, the transgene suppresses a gene early in the oil biosynthetic pathway and a gene late in the oil biosynthetic pathway. In some embodiments of the invention, the seed-oil suppressing transgene is a linear arrangement of partial sense and antisense sequences from a gene that is early in the seed-oil biosynthesis pathway and from a gene that is late in the seed-oil biosynthesis pathway, for example, the CA gene and/or the ACCase gene (early) and the LPAT gene and/or the DGAT gene (late) designed to create RNA interference. The seed-oil suppressing gene may be a cosuppression directing nucleic acid, an antisense nucleic acid, a nucleic acid that encodes an immunomodulation protein, a nucleic acid that encodes a ribozyme, a nucleic acid that encodes a transcription factor suppressor or a nucleic acid that encodes an RNAi sequence.

In some embodiments of the invention, the transgene is operatively linked to a constitutive promoter, such as the 35S promoter from cauliflower mosaic virus, the maize ubiquitin promoter, the peanut chlorotic streak caulimovirus promoter, a Chlorella virus methyltransferase gene promoter, the full-length transcript promoter form figwort mosaic virus, the rice actin promoter, PEMU promoter, MAS promoter, the maize H3 histone promoter or an Agrobacterium gene promoter. The transgene may be operatively linked to a seed-specific promoter such as the cotton alpha-globulin promoter, the napin gene promoter, the soybean alpha-conglycinin gene promoter, the soybean beta-conglycinin gene promoter or the soybean lectin promoter. In some embodiments of the invention, the said seed-specific promoter is generated by operable linkage of a genetic element that directs seed-specific expression to a core promoter sequence.

In a preferred embodiment of the invention, the promoter is activated by application of an external stimulus such that the seed-oil suppressing gene is expressed in the presence of said external stimulus. Exemplary external stimuli include, but are not limited to copper, a benzenesulfonamide herbicide safener, a glucocorticosteroid hormone, estradiol and ecdysterodial activity. In some embodiments of the invention, expression of the seed-oil suppressing gene, after activation, continues to be expressed in the absence of said external stimulus. The seed-oil suppressing gene may be operably linked to an inducible promoter or a repressible promoter, such as, for example the promoter from the ACE1 system, the promoter of the maize Intron 2 gene, the promoter of the Tet repressor from Tn10, the phosphate-deficiency responsive promoter from a phosphate-starvation responsive beta-glucosidase gene from Arabidopsis, the synthetic promoter containing a 235 bp sulfur deficiency response element from a soybean beta-conglycinin gene linked to a 35S core promoter sequence, the inducible promoter from a steroid hormone gene the transcriptional activity of which is induced by a glucocorticosteroid hormone and XVE. The promoter may be a seed-specific promoter, such as the cotton alpha-globulin promoter (AGP).

In some embodiments of the invention, the reduced seed-oil content plant comprises an excisable blocking sequence that prevents expression of said seed-oil suppressing gene. Preferably, the seed-oil content of said plant is reduced to a level of 1% to 17% of the fuzzy whole seed weight. Preferably, stable pools of sucrose are generated in the plant that are available to increase, in a sustained fashion, the production of commercially valuable cellulosic, starch or protein macromolecules.

The invention provides, in some embodiments, a method for making a reduced seed-oil content plant as described above, which comprises transfecting a plant cell with a transgene that suppresses a gene selected from the group consisting of carbonic anhydrase, ACCase, lysophosphatidic acid acyltransferase, diacylglycerol acyltransferase, oleosin and any combination thereof under control of a plant-active promoter; and regenerating a whole plant from the plant cell. The seed-oil suppressing gene may be selected from the group consisting of a cosuppression directing nucleic acid, an antisense nucleic acid, a nucleic acid that encodes an immunomodulation protein, a nucleic acid that encodes a ribozyme, a nucleic acid that encodes a transcription factor suppressor and a nucleic acid that encodes an RNAi sequence. The promoter may be a constitutive promoter, such as the 35S promoter from cauliflower mosaic virus, the maize ubiquitin promoter, the peanut chlorotic streak caulimovirus promoter, a Chlorella virus methyltransferase gene promoter, the full-length transcript promoter form figwort mosaic virus, the rice actin promoter, PEMU promoter, MAS promoter, the maize H3 histone promoter or an Agrobacterium gene promoter. Preferably, the promoter is a seed-specific promoter, such as the cotton alpha-globulin promoter, the napin gene promoter, the soybean alpha-conglycinin gene promoter, the soybean beta-conglycinin gene promoter or the soybean lectin promoter. In some embodiments of the invention, the seed-specific promoter is generated by operable linkage of a genetic element that directs seed-specific expression to a core promoter sequence, and may be activated by application of an external stimulus such that the seed-oil suppressing gene is expressed in the presence of the external stimulus. Exemplary external stimuli include, but are not limited to, copper, a benzenesulfonamide herbicide safener, a glucocorticosteroid hormone, estradiol and ecdysterodial activity.

In some embodiments of the invention, the seed-oil suppressing gene, after activation, continues to be expressed in the absence of said external stimulus. The promoter may be an inducible or repressible promoter, such as, for example, the promoter from the ACE1 system, the promoter of the maize Intron 2 gene, the promoter of the Tet repressor from Tn10, the phosphate-deficiency responsive promoter from a phosphate-starvation responsive beta-glucosidae gene from Arabidopsis, the synthetic promoter containing a 235 bp sulfur deficiency response element from a soybean beta-conglycinin gene linked to a 35S core promoter sequence, the inducible promoter from a steroid hormone gene the transcriptional activity of which is induced by a glucocorticosteroid hormone and XVE. The promoter may be a seed-specific promoter such as the cotton alpha-globulin promoter (AGP).

Preferably, in methods according to the invention, the seed-oil content of said plant is reduced to a level of 1% to 17% or the fuzzy whole seed weight. Also, preferably, stable pools of sucrose are generated in the plant that are available to increase, in a sustained fashion, the production of commercially valuable cellulosic, starch or protein macromolecules.

The invention also provides, in some embodiments, a breeding method for producing an enhanced yield self-pollinating plant that contains a yield enhancing gene, which comprises providing an elite recurrent parent plant; providing a donor parent plant that contains said yield enhancing gene and that contains at least one phenotypic trait; crossing said elite recurrent parent plant with said donor parent plant to produce an F1 progeny plant; crossing said F1 progeny plant with said elite recurrent parent plant to produce a BC1F1 progeny plant that contains said yield enhancing gene; self-pollinating said BC1F1 progeny plant to produce a BC1F2 progeny plant that contains said yield enhancing gene; self-pollinating said BC1F2 progeny plant to produce BC1F2:3 plants that contain said yield enhancing gene; self-pollinating said BC1F2:3 plants; screening said BC1F2:3 plants for zygosity of said yield enhancing gene; collecting the seed of said BC1F2:3 plants that are homozygous for said yield enhancing gene, which is BC1F2:4 seed; and germinating said seed to produce an enhanced yield self-pollinating plant that contains said yield-enhancing gene. Screening can be performed by producing a progeny row and observing a phenotypic trait characteristic of the yield enhancing gene or by molecular biology methods such as PCR or by assay for the expression product of the yield enhancing gene in this or any of the embodiments of the invention.

The invention further provides embodiments which include a breeding method for producing an enhanced yield self-pollinating plant that contains a yield enhancing gene, which comprises providing an elite recurrent parent plant; providing a donor parent plant that contains said yield enhancing gene and that contains at least one phenotypic trait; crossing said elite recurrent parent plant with said donor parent plant to produce an F1 progeny plant; crossing said F1 progeny plant with said elite recurrent parent plant to produce a BC1F1 progeny plant that contains said yield enhancing gene; self-pollinating said BC1F1 progeny plant to produce a BC1F2 progeny plant that contains said yield enhancing gene; self-pollinating said BC1F2 progeny plant to produce BC1F2:3 plants that contain said yield enhancing gene; self-pollinating said BC1F2:3 plants; screening said BC1F2:3 plants for zygosity of said yield enhancing gene; collecting seed of said BC1F2:3 plants that contain said yield enhancing gene, which is BC1F3:4 seed; germinating said BC1F3:4 seed to produce BC1F3:4 plants; self-pollinating said BC1F3:4 plants; screening said BC1F3:4 plants for zygosity of said yield enhancing gene; collecting seed of said BC1F3:4 plants that are homozygous for said yield enhancing gene, which is BC1F3:5 seed; and germinating said seed to produce an enhanced yield self-pollinating plant that contains said yield enhancing gene.

In yet a further embodiment, the invention provides a breeding method for producing an enhanced yield self-pollinating plant that contains a yield enhancing gene, which comprises providing an elite recurrent parent plant; providing a donor parent plant that contains said yield enhancing gene and that contains at least one phenotypic trait; crossing said elite recurrent parent plant with said donor parent plant to produce an F1 progeny plant; crossing said F1 progeny plant with said elite recurrent parent plant to produce a BC1F1 progeny plant that contains said yield enhancing gene; self-pollinating said BC1F1 progeny plant to produce a BC1F2 progeny plant that contains said yield enhancing gene; self-pollinating said BC1F2 progeny plant to produce BC1F2:3 plants that contain said yield enhancing gene; self-pollinating said BC1F2:3 plants; screening said BC1F2:3 plants for zygosity of said yield enhancing gene; collecting seed of said BC1F2:3 plants that contain said yield enhancing gene which is BC1F3:4 seed; germinating said BC1F3:4 seed to produce BC1F3:4 plants; self-pollinating said BC1F3:4 plants; screening said BC1F3:4 plants for zygosity of said yield enhancing gene; collecting seed of said BC1F3:4 plants that contain said yield enhancing gene, which is BC1F4:5 seed; germinating said BC1F4:5 seed to produce BC1F4:5 plants; self-pollinating said BC1F4:5 plants; screening said BC1F4:5 plants for zygosity of said yield enhancing gene; collecting seed of said BC1F4:5 plants that are homozygous for said yield enhancing gene, which is BC1F4:6 seed; and germinating said seed to produce an enhanced yield self-pollinating plant that contains said yield enhancing gene.

In further embodiments, the method further comprises repeating the steps of screening the plants for zygosity of the yield enhancing gene; self-pollinating the plants; collecting the seed of the plants that contain said yield enhancing gene; germinating said BC1F4:5 seed to produce BC1F4:5 plants; self-pollinating the plants; screening the plants for zygosity of said yield enhancing gene; collecting seed of the plants that are homozygous for said yield enhancing gene; and germinating the seed to produce plants for generations of heterozygous progeny plants subsequent to BC1F4:5.

In preferred embodiments of the invention, the yield enhancing trait is selected from the group consisting of seed-oil suppression, delayed leaf senescence, enhanced leaf photosynthesis, enhanced leaf production of sucrose, enhance leaf export of sucrose, enhanced translocation of sucrose in the plant vasculature, reduced plant respiratory losses, reduced plant photorespiratory losses, reduced carbohydrate use in non-fruit plant tissue, enhanced movement of sucrose into the desired plant organ or tissue, and any combination thereof. The phenotypic trait may be selected from the group consisting of dwarfing, short stature, more determinate growth habit, precocious flowering, intense flowering, rapid fruit development, medium to large seeds, large bolls, high fruit retention, high lint percent, low micronaire, cluster fruiting, insect protection, and any combination thereof.

Elite recurrent parent plants suitable for use in the invention preferably are selected for a quality selected from the group consisting of yield, adaptation, fiber quality, agronomic performance and transgenic traits. The yield enhancing gene may be a mutant allele of a gene naturally occurring in said plant or a transgene. The donor parent plant may be produced by directly transforming a recurrent plant containing at least one phenotypic trait with a yield enhancing gene, by crossing a yield enhancing gene donor plant with a recurrent plant containing at least one phenotypic trait and selecting progeny plants that contain both the yield enhancing gene and the phenotypic trait(s) or by crossing and backcrossing a yield enhancing gene donor plant with a recurrent plant containing at least one phenotypic trait and selecting progeny plants that contain both the yield enhancing gene and the phenotypic trait(s).

### BRIEF DESCRIPTION OF THE FIGURES

FIG. 1 shows the relationship between Lint Yield and Oil Percent in 21 cotton varieties from 1989 through 2001 (Rayburn et al., 1989-2001).

FIG. 2 shows the relationship between Lint Yield and Lint Percent in 21 cotton varieties from 1989 through 2001 (Rayburn et al., 1989-2001).

FIG. 3 shows the relationship between Lint Percent and Oil Percent in 21 cotton varieties from 1989 through 2001 (Rayburn et al., 1989-2001).

FIG. 4 shows the relationship between Lint Yield (as determined from multiple paired comparisons) and Oil Percent in 19 cotton varieties from 2002.

### DETAILED DESCRIPTION OF THE INVENTION

Yield Enhancement Technology (YET) in field crops has been an active focus of recent research in plant biology. Although yield enhancement research in cotton has been limited by the difficulty of genetic manipulation in cotton and its complex harvestable product, the chance of success in cotton is greater than in other field crops because physiological optimization for yield within modem cotton cultivars is limited. During most of cotton's domestication, up until the 1950's, cotton was cultivated around the world as a full season crop or a perennial shrub without insecticide protection. Despite numerous host plant resistance mechanisms in cotton, percent fruit retention, and by extension sucrose utilization in fiber development, was low. However, with modern chemical and biotechnological solutions for insect pests, combined with reduced season-length production strategies, percent boll set has substantially improved and the rate of sucrose utilization in fiber development has increased. Yield in modern cotton fields is now limited by sucrose supply rather than fruit retention. This limitation of fiber yield by sucrose is evidenced in the 35% increase in yield response to increased leaf sucrose production with elevated CO₂ (Evans, 1990; Kimball, 1983) and the global appearance of boll-accumulation-associated syndromes (bronze wilt, potassium deficiency, dead fiber, and premature senescence) in high yielding fields and cultivars as the plant carbohydrate supply is exhausted.

The biosynthesis of cottonseed-oil reduces the supply of sucrose available for biosynthesis of fiber and additional vegetative growth due to: (a) the common source of carbon (phloem-transported sucrose) for both seed-oil (primarily TAG) and cellulose, (b) the temporal overlap on a whole plant basis in sucrose utilization for both oil and cellulose, and (c) the relative energetic inefficiency of the seed-oil biosynthetic pathway compared with cellulose biosynthesis. As a result of sucrose depletion during the boll maturation phase, new vegetative tissue and fiber development ceases during the period referred to as “cutout” (Kerby et al., 1993). As the sucrose supply declines, new root production slows first, then shoot tissue production slows and finally boll accumulation stops. Fiber yield is curtailed both directly due to the diversion of sucrose to oil biosynthesis and indirectly from the decline in photosynthesis and soil nutrient uptake that results from the aging of the leaf and root tissues when new shoot and root production slows. Wullschleger and Oosterhuis, 1990; Kerby et al., 1987.

A method to suppress TAG biosynthesis in cottonseed would delay cutout, sustain photosynthesis and soil nutrient uptake and provide an expanded sucrose supply for fiber development. Whole plant modeling has demonstrated in silico the yield benefit derived from delayed cutout (Landivar et al., 1983). Since TAG requires substantially more metabolic energy to produce than cellulose does and the mass of seed is greater than the mass of fiber, a slight reduction in TAG could result in an economically significant increase in fiber yield if the sucrose supply is properly channeled into fiber production and not excess shoot or root growth. This channeling of excess sucrose into fiber, without excess vegetative growth, will require modifications to traditional agronomic and plant breeding programs.

Considering that seed-oil, primarily TAG, is used in the developing cotton seedling as a source of energy and carbon for new growth (Cothren, 1999; Shrestha et al., 2002), extreme elimination of TAG biosynthesis in seed, which would generate the largest fiber yield enhancement, could limit seedling growth. A cotton variety, DP 5415, with small seeds and low oil content (Seed Index of 8.7 g per 100 seeds and Oil Percent of 18%, USDA-ARS) produces smaller and less vigorous seedlings. In Arabidopsis mutants, a 25% reduction in seed-oil content impairs germination under cool or saline conditions (Lu and Hills, 2002). Other Arabidopsis mutants, peroxisome defective (ped1, 2 and 3) and isocitrate lyase knockouts (icl-1 and icl-2), with reduced utilization of fatty acids, generated viable seeds which failed to develop normal seedlings under less than optimum light and nutritional conditions (Hayashi et al., 1998; Eastmond et al., 2000).

Thus, TAG suppression in a cultivar for commercial uses would have to be controlled or regulated by a plant gene expression system to allow the production of a seed crop without cottonseed-oil suppression, but which allows these viable seeds to produce a commercial fiber crop with maximum fiber yield enhancement. Such a mechanism would allow cotton farmers both to plant vigorous seed and harvest higher fiber yields, since the supply of sucrose not diverted to TAG would be used by the plant to produce fiber. TAG suppression in the seed is not expected to impact developing fiber negatively since TAGs are neutral, anhydrous lipids produced and stored in metabolically-isolated oil bodies after peak fiber development in the same boll. Indirect effects on fiber and embryo metabolism may occur due to the increased supply of acetyl CoA and precursors, including sucrose, available for non-oil biosynthetic pathways on a whole plant basis.

The inverse relationship between seed-oil and the other seed constituents, protein and carbohydrate (starch or cellulose), has been a challenge to plant breeding in corn and soybeans, where genetic gain for combined seed yield and oil content have been difficult (Kumar et al., 2000) and where genetic gain for one constituent has resulted in genetic loss in other constituents (Dudley, 1992). Genetic selection in corn and soybeans for high oil or high protein negatively impacts grain yield (Dudley, 1976; Burton, 1987). In Arabidopsis mutants that have low seed-oil content, seed carbohydrates such as starch and sucrose increase. Tobacco leaves transgenically modified to overexpress the lipid biosynthetic enzyme, acetyl-CoA carboxylase (ACCase), have about 20% higher lipid content and about 20% lower starch content compared with wild type leaves (Madoka et al., 2002). Potatoes transgenically altered to suppress starch biosynthesis using antisense have increased sugar and protein content (Willmitzer et al., 2001).

Expanded sucrose supply for fiber development (and thus yield enhancement) could be generated from one of the following four approaches: (1) increasing photosynthetic efficiency, (2) extending the duration of leaf photosynthesis, (3) avoiding damage to leaf photosynthetic machinery, and (4) reducing sucrose diverted to plant tissues or products that are less valuable than fiber. To achieve any of the first three of these approaches probably would require complex multi-genic modification; the fourth approach can be achieved by cottonseed oil suppression. The combination of new technology to modify seed-oil combined with the recognition of the greater economic value of the cotton fiber compared with the cotton oil (45 cents per pound of fiber compared with 18 cents per pound of cottonseed oil; USDA-AMS, 2002) provides a beneficial and useful method to achieve yield enhancement in cotton.

The biosynthesis of oil has multiple regulatory and enzymatic points that are potential targets for downregulation by cosuppression (or gene silencing) (Waterhouse et al., 2002), antisense (Cornelissen and Vandewiele, 1989), immunomodulation (Jobling et al., 2002), ribozymes (Cotten and Bimstiel, 1989), transcription factor suppression (Guan et al., 2002), RNAi strategies (Fire et al., 1998; Chuang and Meyerowitz, 2000) or other methods known in the art. These targets include, but are not limited to carbonic anhydrase (CA) (Hoang and Chapman, 2002a), ACCase (Reverdatto et al., 1999), lysophosphatidic acid acyltransferase (LPAT) (Ohlrogge and Jaworski, 1997), diacyglycerol acyltransferase (Ohlrogge and Jaworski, 1997; Roesler et al., 1997), and oleosin expression (Sarmiento et al., 1997).

Hoang and Chapman (2002a) have discussed carbonic anhydrase (CA) as a potential target for lipid biosynthesis suppression in the cotton variety Paymaster HS 26. In plant fatty acid biosynthesis, HCO₃- is incorporated by acetyl-CoA carboxylase (ACCase) while CO₂ is released by fatty acid synthase (FAS). Acetyl-CoA provides the carbon source for fatty acid biosynthesis and production of TAG. Because the uncatalyzed interconversion of HCO₃- and CO₂ is slow, CA activity increases 15-fold from 25 days to 40 days post anthesis in cottonseeds to support oil biosynthesis (Hoang and Chapman, 2002a). Furthermore, CA inhibitors applied to cotton embryos and antisense suppression of CA in tobacco both reduced oil biosynthesis. Fatty acid availability in embryos has been identified as a limiting factor in TAG accumulation (Bao and Ohlrogge, 1999). A novel CA gene in cottonseeds has been identified (Wessler et al., 2002). The level and/or timing of expression of the CA gene are viable targets to suppress cottonseed oil without suppressing the other functions of CA in non-seed tissue.

ACCase is the first committed step in fatty acid biosynthesis. Two forms of ACCase occur in dicotyledonous plants; the homodimeric form (HOM-ACCase) occurs in the cytosol, while the heteromeric form (HET-ACCase) occurs in the plastids where the fatty acids are synthesized. These fatty acids later are incorporated into TAG oil bodies via the endoplasmic reticulum. ACCase is responsible for the capture and complexing of HCO₃- with acetyl-Coenzyme A (CoA), producing Malony-CoA which is subsequently used in the 2-carbon step elongation of fatty acids. Acetyl-CoA is a common intermediate in many cellular metabolic pathways and is found in plastids, cytoplasm, mitochondria, and peroxisomes/glyoxysomes. Three of the four HET-ACCase subunits in dicots are coded by nuclear genes (Somerville et al., 2000; Sasaki et al., 2001) and have been characterized, sequenced and cloned in pea and soybean (Shorrosh et al., 1996; Reverdatto et al., 1999). These genes therefore are suitable and appropriate targets for cosuppression, antisense, ribozyme, transcription factor suppression or nuclear transgene RNA inhibition methods.

In the grass family (Poaceae), homodimeric forms of ACCase are found in both the cytosol and plastid. The grass plastid-localized HOM-ACCase is the site of inhibition for the cyclohexenone herbicides, and has been sequenced in several plant species (Reverdatto et al., 1999). Targeting of the Arabidopsis HOM-ACCase to the Brassica napus plastid resulted in a 6% increase in total seed-oil content (Roesler et al., 1997). Tobacco leaves transgenically modified to overexpress ACCase have about 20% higher lipid content and about 20% lower starch content compared to wild type leaves (Madoka et al., 2002). Antisense suppression of the Brassica napus HOM-ACCase reduced seed lipid to 66 and 86% of wild type while increasing sucrose content by 35% compared to wild type (Sellwood et al., 2000). The coordinate expression of ACCase with other lipid biosynthetic components suggests that the accumulation of ACCase mRNA is a key regulatory point in lipid biosynthesis (O'Hara et al., 2002). Positioned at the beginning of lipid biosynthesis, ACCase is a tightly regulated enzyme in plants. This enzyme therefore is a suitable target for downregulation with seed-specific expression of sense, antisense, ribozyme, transcription factor suppression or RNA interference to permit oil suppression without adverse overproduction of precursors or inhibition of plant growth according to an embodiment of this invention.

Diacyglycerol acyltransferase (DGAT) catalyzes the final step in the biosynthesis of TAG, using diacylglycerol and a fatty acid CoA as substrates. Its primary protein sequences in Arabidopsis thaliana, Nicotiana tabacum and Brassica napis recently have been elucidated and are 90% homologous at the amino acid level (Bouvier-Nave et al., 2002; Zou et al., 1999; Hobbs et al., 1999; Nykiforuk et al., 1999; Jako et al., 2001). DGAT is unique to TAG biosynthesis and thus its downregulation can impact TAG accumulation (Katavic et al., 1995) without curtailing other metabolic pathways. Genes coding for DGAT have been cloned (Hobbs et al., 1999; Lardizobal, 2001). The ethyl methanesulfonate (EMS)-derived insertional mutant, tag 1-1, in Arabidopsis thaliana had DGAT activity and seed-oil content approximately 75% of the wild type (Jako et al., 2001), with nonsignificant effects on protein or seed size. Sucrose was elevated by 55% in tag 1-1 mutant seeds compared to wild type (Lu and Hills, 2002). Another Arabidopsis DGAT mutant, tag 1-2, develops seed-oil contents approximately 55% of wild type and germination and cotyledon emergence under favorable conditions (23° C.) 45% of wild type. Germination of the tag 1-1 mutant was comparable with wild type under favorable conditions. Under cold conditions (4° C.), tag 1-1 and tag1-2 germination was reduced to 45% and 0%, respectively, from 100% and 85% in wild type (Lu and Hills, 2002). Sensitivity to salt was similarly related to low TAG content (Lu and Hills, 2002). In Arabidopsis thaliana, tobacco, and Brassica napus, overexpression of DGAT using a seed-specific promoter heritably and significantly increased seed-oil content, seed weight, and seed yield with minimal changes in the fatty acid composition of the seed-oil (Jako et al., 2002; Bouvier-Nave et al., 2002).

An earlier enzyme in the biosynthetic pathway of TAG, lysophosphatidic acid acyltransferase (LPAT), also regulates TAG production in Arabidopsis thaliana and Brassica napus (Zou et al., 1997). The sequence of the penultimate enzyme in the formation of TAG, phosphatidic acid phosphatase (PAP), has been reported (Lassner, 2002). PAP dephosphorylates the sn-3 position of phosphatidic acid to form sn-1,2-diacylglycerol, the precursor of TAG. Lassner (2002) has claimed that use of sense and antisense sequences of PAP to increase and decrease TAG in corn and soybeans produces plants with altered lipid composition and total lipid levels. Although most of the current work in oil biosynthetic manipulation has been focused on increasing oil content, such as the work with LPAT, downregulation of a pathway generally is considered to require less genetic manipulation than upregulation, where the biosynthesis of precursor molecules also must be upregulated (Taylor, 1998).

Oleosins are multidomain proteins embedded in the monolayer lipid surface of oil bodies. Oleosins have a hydrophobic domain that protrudes into the TAG core of the oil body and amphiphatic domains that reside on the surface of the oil body or protrude from it. The accumulation of TAG and oleosins is synchronous in developing embryos and highly correlated in some plant species (Tzen et al., 1993). Since oleosins are expressed in seed and used only in oil bodies (Sarmiento et al., 1997), downregulation of oleosins is a potential target for significant suppression of cottonseed oil storage. To minimize accumulation of TAG precursors, suppression of oleosins may be and preferably is combined with inhibition of an enzyme early in the lipid biosynthetic pathway, such as CA or ACCase. Near total elimination of oleosins in seed, combined with optimum suppression of CA or ACCase, according to an embodiment of this invention, can produce sufficient lipid for non-TAG uses (and thus permit normal seed and fiber development) while avoiding precursor build-up from the complete suppression of oil bodies.

Cosuppression (or gene silencing), antisense, immunomodulation, ribozyme, transcription factor suppression or RNAi strategies are all known per se to those skilled in the art. Therefore, these strategies are easily adapted to optimize the embodiments of this invention. Preferably, genes for the inhibition of cottonseed oil biosynthetic enzymes would use seed-specific promoters that have negligible activity in non-seed tissue. Use of seed-specific promoters would minimize the chance for metabolic disruption in non-seed tissues and lessen the metabolic cost associated with constitutive expression or over expression of transgenic proteins. An alpha-globulin promoter (AGP) from cotton recently has been identified and shown to have a high level of seed-specific activity in cotton (Sunilkumar et al., 2002) without measurable non-seed expression. Therefore, this seed-specific promoter is preferred for expression of transgenes in embodiments of the invention involving transgene expression.

Seed-specific promoters in specific enzyme suppression (Chapman et al., 2001) and RNAi strategies (Liu et al., 2002) to inhibit seed fatty acid modification enzymes have led to successful manipulation of seed-oil quality parameters in cotton. These research programs have focused on inhibiting the activity of the oil desaturases that produce linoleic from oleic and steric oil for the purpose of enhanced nutritional and cooking qualities. Seed-specific promoters have not been reported for use in cosuppression (or gene silencing), antisense, immunomodulation, ribozyme, transcription factor suppression or RNAi strategies for the suppression of oil biosynthesis for enhanced fiber yield.

Whether CA, ACCase, LPAT, DGAT, and/or oleosin genes are the target for cottonseed oil suppression, the selection of optimum gene targets or sets of gene targets for downregulation or delayed expression using cosuppression (or gene silencing), antisense, immunomodulation, ribozyme, transcription factor suppression or RNAi strategies depends upon the objective level of cottonseed oil suppression. A gene expression control system can achieve a high degree of oil suppression to maximize fiber yield enhancement. Only partial oil suppression would be required for non-gene-regulated systems that are functional in both the seed production generation and the subsequent fiber production generation.

In cotton embryos, a 90% CA suppression generates only a 50% suppression of oil biosynthesis, ostensibly due to the non-enzymatic interconversion of HCO₃- and CO₂ that allows some production of acetyl CoA for oil biosynthesis (Hoang et al., 2002a). Suppression of additional enzymes in the TAG biosynthetic pathway could be designed into a system for more complete inhibition of stored seed-oil. Since oil is a significant source of energy and carbon for early seedling growth, a very high degree of cottonseed oil suppression may negatively impact seedling growth and therefore subsequent yield; reduced oil content planting seed exhibit reduced seedling vigor and germination of immature and low density cotton (Hopper et al., 1999; Cherry et al., 1986). Cotton cultivars selected under a wide range of conditions have a narrow range of seed oil content, 18% to 22% w/w (Rayburn et a., 1989 through 2001), evidencing oil's key role in providing metabolic energy for seedling growth. To achieve the highest level of fiber yield enhancement from cottonseed oil suppression and still allow propagation of high vigor seed for planting and sale, gene expression preferably is regulated in various generations that are grown for planting seed or fiber production.

Oliver et al. proposed a mechanism to regulate gene expression using an excisable block between a late embryogenesis activated (LEA) promoter and a seed germination inhibitor. See U.S. Pat. Nos. 5,723,765; 5,925,808; 5,977,441 and Oliver and Velten (2001). This mechanism subsequently was demonstrated to be fully operational in model crops. By replacing the LEA promoter with the AGP promoter and the seed germination inhibitor with cosuppression (or gene silencing), antisense, immunomodulation, ribozyme, transcription factor suppression or RNAi genetic elements that inhibit TAG biosynthesis and/or TAG storage, this mechanism is used in an embodiment of the invention to generate high vigor cotton seed which, when planted for fiber yield, produces plants with a high degree of cottonseed oil suppression. Seed produced is viable but low in TAG and thus has reduced capacity to establish a seedling under field conditions. If suppression at multiple sites in the TAG biosynthetic pathway are desired to achieve maximum fiber yield enhancement while maintaining a supply of fatty acids for normal seed development, multiple cosuppression (or gene silencing), antisense, immunomodulation, ribozyme, transcription factor suppression or RNAi directed inhibition sequences can be operably linked to one promoter (Abbott, 2002).

Once the genetic traits for both high and low levels of TAG suppression in cottonseed have been transformed into cotton, the testing and introgression of this trait into elite cotton cultivars with fiber yield enhancement is conducted. Traditional methods of trait selection in transformed and regenerated cotton plants can be employed. DNA, RNA, or protein selection for the trait can occur in callus or regenerated tissue. TAG concentration is determined for selection in the T1 and/or subsequent generations, since the oil suppression trait of interest will be expressed only in seed.

Although TAG levels can be evaluated in transformed seed, the resulting fiber yield enhancement from cottonseed oil suppression technology is evaluated best in elite germplasm. Gene expression of some yield enhancing technology methods will fit optimization curves having too little or too much effect. Thus it may be necessary for some embodiments to carry out multiple insertion events having varying levels of gene expression, further into the testing and introgression program, to make final event selection decisions once introgressed into elite germplasm. This type of adjustment to expression levels is routine.

Current transgenic products can generate minor pleiotrophic effects in some germplasm, such as increased storm resistance or earlier maturity. These are small compared with the potential pleiotrophic effects of yield enhancing technology that by their nature can alter maturity, determinacy, fiber quality, plant height, canopy architecture, disease susceptibility, response to plant growth regulators and harvest aids, etc. The potential for interaction between a yield-enhancing trait (such as TAG suppression) and the base germplasm requires novel breeding methods that take this potential interaction into account to prevent undesired effects in the whole plant (such as too great an increase in vegetative growth) which may occur when a yield enhancing gene such as a seed-oil suppressing (SOS) gene is expressed. The breeding methods described as follows are appropriate and preferred with embodiments of the invention, but can be used for much broader applications.

A donor parent containing the transgenic yield enhancing trait such as TAG suppression, or any yield enhancing trait whether recognized only phenotypically or due to expression of an identified gene (including transgenes), can be produced using traditional crossing and selection methods to also possess a unique phenotype or set of phenotypes (such as dwarfing, large bolls and precocious flowering, for example, for cotton) that contribute multiple phenotypic traits, any one or any group of which potentially can enhance the effect of the transgenic yield-enhancing trait when expressed in a particular genetic background. Any phenotypic trait or group of phenotypic traits may be used with the methods of the invention. Therefore, the term “phenotypic trait” refers herein to any phenotypic characteristic of a plant known in the art to those of skill in plant breeding or any gene the expression of which produces such a phenotypic characteristic. The term “yield enhancing trait” refers to any phenotypic or genotypic characteristic that results in increased yield of a desired plant product in plants carrying the yield enhancing trait. The term includes any gene or transgene which, when expressed, results in a plant with enhanced yield of the desired plant product. Examples of “yield enhancing traits” include, but are not limited to, seed-oil suppression, delayed leaf senescence, enhanced leaf photosynthesis, enhanced leaf production of sucrose, enhance leaf export of sucrose, enhanced translocation of sucrose in the plant vasculature, reduced plant respiratory losses, reduced plant photorespiratory losses, reduced carbohydrate use in non-fruit plant tissue, enhanced movement of sucrose into the desired plant organ or tissue, or any combination thereof. Examples of “phenotypic traits” include, but are not limited to, dwarfing, short stature, more determinate growth habit, precocious flowering, intense flowering, rapid fruit development, medium to large seeds, large bolls, high fruit retention, high lint percent, low micronaire, cluster fruiting, insect protection, or any combination thereof.

The donor parent, which contains the yield enhancing trait, for example an SOS transgene, and a number of different phenotypic traits, is crossed with elite recurrent parents that previously had been selected for yield, fiber quality and/or agronomic performance. Progeny from crosses with different elite recurrent parents, containing different sets of the multiple phenotypic traits of the donor parent, can be selected using methods well known in the art for the optimal combination of traits which allow the enhanced yield transgene to produce the best results.

In addition, this donor parent may be used for limited backcrossing with elite recurrent parents, which transfers the yield-enhancing transgenetic trait along with a subset of the multiple phenotypic traits from the donor parent into the elite recurrent parent germplasm. Subsequent selection of optimal combinations can optimize the combinations of traits which allow maximum performance of the yield enhancing trait. Limited backcrossing initially involves crossing the donor parent with an elite recurrent parent and backcrossing once to the same elite recurrent parent, then selection of lines in the BC1F2, BC1F3, BC1F4, BC1F5 or higher generation. The limited backcrossing is performed to ensure that the derived lines contain (at least substantially) the genetic compliment of the elite recurrent parent along with the yield-enhancing trait and either none, some or all of the potentially beneficial enhancing phenotypic traits contributed by the donor parent.

In subsequent generations, an array of BC1F2, BC1F3, BC1F4, BC1F5 or higher derived lines are evaluated to allow identification of specific phenotypic traits and combinations of traits which enhance the effect of the yield-enhancing trait in any particular elite recurrent parent background. Yield and agronomic optimization of a particular yield-enhancing trait in different elite recurrent parent backgrounds can require different combinations of multiple phenotypic traits derived from the donor parent for maximum performance. Traditional methods of plant breeding, therefore, can be used to produce the best combination of traits for the desired transgenic trait and the desired elite recurrent parent. Preferred phenotypic traits for use in these methods with a seed-oil suppressing transgene donor include, but are not limited to dwarfing, short stature, more determinate growth habit, precocious flowering, intense flowering, rapid fruit development, medium- to large-seeded, large bolls and high fruit retention. Any traits known to breeders can be used with the transgene of choice. Plant breeders have used these methods as a matter of routine to produce lines with particular desired traits, therefore it is considered normal practice.

In geographical regions with frequent cotton insect pest occurrence that damage fruiting sites, squares, and bolls, the cottonseed oil suppression trait optimally is combined with efficacious broad spectrum insect control strategies to minimize fruit loss that could lead to excess vegetative growth, plant height and delayed maturity. One optimal insect control strategy for combining with cottonseed oil suppression traits is multiple genes derived from Bacillus thuringiensis. These multiple gene traits are being developed by several sources (Pellow et al., 2002; Greenplate et al., 2002) and are known in the art. Other genes with broad spectrum insect control also are being developed and in some preferred embodiments one or more of these are combined with the cottonseed oil suppression trait to provide yield enhancement that is of substantial benefit to most farmers. Placing this double technology in elite germplasm with early and prolific square production would further enhance farmer value.

This invention relates to methods for generating plants wherein the seed-oil content is reduced, thereby increasing the supply of sucrose for protein and carbohydrate (including cellulose and starch) production within the seed. By controlling the expression of genes that affect the plant phenotype, it is possible to grow plants under one set of conditions or in one environment where one phenotype is advantageous and then alter the environment, either directly or indirectly by moving the plant or planting its seed under another set of conditions where a different phenotype is advantageous. This technique has particular utility in agricultural and horticultural applications.

As used in this specification, the term “gene” refers to a segment of DNA which encodes a specific protein or polypeptide, or an RNA. The term “seed-oil suppressing gene,” or “seed-oil suppression gene” or “SOS gene” refers to a segment of DNA the expression of which eliminates, blocks, hinders, interferes with, prevents, or otherwise reduces the biosynthesis or storage of seed-oil. Such seed-oil suppressing genes may include sequences that direct cosuppression, antisense sequences, sequences that encode antibodies, RNAi sequences or ribozymes, or any other DNA sequence with the desired function.

As used in this specification, the term “plant-active promoter” refers to a DNA sequence that directs the transcription of an operably linked DNA sequence in a plant cell. Typically, a promoter is located in the 5′ region of a gene, proximal to the transcriptional start site of a structural gene. A plant-active promoter can be of viral, bacterial, fungal, animal, or plant origin.

As used in this specification, the term “constitutive promoter” refers to a plant-active promoter that effects the transcription of a DNA sequence, irrespective of temporal, developmental, hormonal, chemical or environmental conditions, for example in the absence of a traditional ligand.

As used in this specification, the term “seed-specific promoter” refers to any plant-active promoter that is either active exclusively in the plant seed or active in the plant seed and to a lesser degree in other plant tissues. Seed-specific promoters also can contain an added seed tissue-preferred regulatory region, a nucleotide sequence that directs a higher level of transcription of an associated gene in seed tissues than in some or all other tissues of a plant, with a core plant-active promoter that may be of plant, viral, bacterial, fungal, or animal origin. Seed-specific promoters also can include enhancer elements that confer upon a core promoter seed tissue expression specificity.

The term “enhancer” refers to a DNA regulatory element that can increase the efficiency of transcription or confer tissue specificity, regardless of the distance or orientation of the enhancer relative to the start site of transcription.

As used in this specification, the term “inducible promoter” refers to any promoter the activity of which is altered by the application of, or exposure to, an external stimulus. In most applications, when a promoter is inducible the rate of transcription increases in response to an external stimulus. The external stimulus can be chemical, environmental (physical), or biological in nature.

As used in this specification, the term “cosuppression” refers to the process by which over-expression of an introduced gene results in the downregulation of both the introduced gene and the homologous endogenous gene.

As used in this specification, the term “antisense” refers to a gene or a nucleotide sequence derived thereof which has a homology of more than 50%, preferably more than 80% with a target gene as defined herein, and which is linked to a promoter in the inverse 5′ to 3′ orientation with respect to the target gene.

As used in this specification, the term “RNAi” (RNA interference) refers to suppression of the expression of a target gene by the introduction and expression of sequences that correspond to the whole or part of the target gene and that generate double-stranded RNAs, resulting in target gene specific gene silencing (suppression). Generally such RNAi sequences comprise a linear arrangement of partial sense and antisense DNA sequences, expression of which forms RNA interference.

As used in this specification, the term “ribozyme” refers to an RNA molecule that contains a catalytic center. The term includes RNA enzymes, selfsplicing RNAs, and self-cleaving RNAs. A DNA sequence that encodes a ribozyme is termed a ribozyme gene.

As used in this specification, the term “immunomodulation” refers to the expression of antibody molecules that disrupt the function of specific enzymes.

As used in this specification, the term “transcription factor suppression” refers to suppression or delay of gene transcription by expressing trans acting protein binding molecules that have this effect.

A gene and a promoter are considered to be operably linked if they are on the same strand of DNA, in the same orientation, and are located relative to one another such that the promoter is capable of directing transcription of the gene (i.e. in cis). The presence of intervening DNA sequences within or between the promoter and the gene does not preclude an operable relationship.

A blocking sequence is a DNA sequence of any length that blocks a promoter from effecting expression of a targeted gene.

A specific excision sequence is a DNA sequence that is recognized by a site-specific recombinase.

A recombinase is an enzyme that recognizes a specific excision sequence or set of specific excision sequences and effects the removal of, or otherwise alters, DNA between specific excision sequences.

The present invention involves, in a first embodiment, the identification and isolation of plants, either naturally occurring or artificially generated, that express seed-oil suppression genes (or alleles of those genes) that control seed-oil content in existing elite and primitive germplasms. These genes (or alleles) are referred to as “seed-oil suppression genes,” “seed-oil suppressing genes” or “SOS genes.” In accordance with the first embodiment, seed samples from individual plants or lines from existing plant populations, varieties or race stocks for reduced oil content and increased carbohydrate and/or protein are screened to identify and isolate plants that contain seed-oil suppression alleles. Alternatively, plants containing seed-oil suppression alleles may be identified and isolated from populations generated from mutagenized seed stocks. Mutagenesis of the seed stocks may be performed by any method known in the art.

Once an SOS gene, or any transgene, has been identified, plants, seeds, plant cells or plant tissues advantageously may be screened for presence of the gene. Methods for so doing are known in the art and may include, for example, any of the methods described in Ahmed, (2002) “Detection of Genetically Modified Organisms in Food.” Trends Biotechnol. 20:215-233. These methods are routinely used by those of skill in the art and may be modified to fit the particular conditions. Testing for zygosity of an SOS gene, or any transgene, may be performed using any method known in the art. For example, the methods described in Calloway AS, Abranches R, Scraggs J. Allen GC, Thompson WF (2002) “High Throughput Transgene Copy Number Estimation by Competitive PCR.” Plant Mol. Biol. Reporter 20:265-277 are suitable. Alternatively, zygosity may be determined by planting a progeny row and observing in the progeny whether the trait of interest is homozygous or whether the trait segregates. Generally these methods involve planting a row of progeny from a single plant containing enough progeny to be able to determine statistically whether a particular trait was homozygous, heterozygous or hemizygous in the parent according to well-known Mendelian methods.

The seed-oil content assessment protocols developed and published by the American Oil Chemists' Society are convenient to use for screening purposes, however any convenient method known in the art is contemplated for use with this invention. For example, the protocol Aa 4-38 (AOCS, 2001; the disclosures of which are hereby incorporated by reference) describes the isolation and measurement of cottonseed oil. Seed samples may be collected and assayed from individual plants or lines from elite or primitive germplasm stocks, or both. Alternatively, alleles of endogenous seed-oil content genes that suppress seed-oil biosynthesis and/or storage may be generated within an elite (or other) germplasm by random mutagenesis using techniques well known to those versed in the art. For example, a cotton seed stock can be mutated by exposure to the chemical mutagen ethyl methanesulfonate using the protocol described by Auld et al., 1998, the disclosures of which are hereby incorporated by reference. Alleles of mutated seed-oil content genes that suppress seed-oil biosynthesis and/or storage are identified and isolated as described above for natural seed-oil suppression alleles. Once identified and isolated, the seed-oil suppression alleles can be introduced into elite germplasms for commercial evaluation by the use of standard plant breeding techniques well known to those versed in the art.

The present invention involves, in a second embodiment, the generation of transgenic plants that contain a transgene the expression of which can suppress seed-oil biosynthesis or reduce the seed-oil content within the seed. This transgene may be, but is not limited to, a gene the expression of which results in the suppression of one or more steps in the seed lipid biosynthetic pathway. Such transgenes and groups of nucleic acid sequences also are included in the meaning of the term “seed-oil suppression gene” as defined above. In accordance with a preferred embodiment of this type of transgenic plant, the plant has introduced into it a series of sequences that include a plant-active promoter linked to a seed-oil suppression gene. In the first and second embodiments, the seed-oil suppression gene is expressed in the seed during its development.

In this second embodiment of the invention, a genetic system for seed-oil suppression is generated and introduced into a plant. The seed-oil suppression system advantageously comprises a plant-active promoter operably linked to a seed-oil suppression gene. The plant-active promoter is preferably but not necessarily constitutive or seed-specific in its transcriptional activity. Suitable constitutive promoters include, but are not limited to, the 35S and 19S promoters from the cauliflower mosaic virus (CaMV) genome (Comai et al., 1992., Fraley et al., 1996), the 34S promoter from the figwort mosaic virus (FMV) genome (Comai et al., 2000), the maize ubiquitin promoter (Cigan et al., 1998), the peanut chlorotic streak caulimovirus (PCISV) promoter (Maiti et al., 1998), promoters of Chlorella virus methyltransferase genes (Mitra et al., 1996), the full-length transcript promoter from figwort mosaic virus (FMV) (Rogers, 1995); the rice actin promoter (McElroy et al., 1990), pEMU promoter (Last et al., 1991), MAS (Velten et al., 1984), maize H3 histone (Lepetit et al., 1992; Atanassova et al., 1992), and promoters of various Agrobacterium genes (Gelvin, 1988; Hall et al., 1992; Slightom et al., 1993; Barker et al., 1995). Suitable seed-specific promoters include, but are not limited to, the cotton alpha-globulin promoter (AGP, Sunilkumar et al., 2002), the napin gene promoter (Kridl et al., 1991), soybean alpha- and beta-conglycinin genes (Barker et al., 1988; Chen et al., 1988; Lessard et al., 1993), and soybean lectin promoter (Townsend and Llewellyn, 2002). Seed-specific promoters also can be generated by operable linkage of genetic elements that direct seed-specific expression to core promoter sequences. Such synthetic seed-specific promoters include, but are not limited to, the use of a concatemerized B-Box element from a 2S napin gene promoter to a 35S minimal promoter sequence (Rask et al., 1998; Ezcurra et al., 1999), the addition of a G-Box element from the strictosidine synthase gene from Catharanthus roseus (Ouwerkerk and Memelink, 1999), and the linkage of a 68 bp seed specific enhancer (SSE) element from a beta-phaseolin gene to a 35S basal promoter (−64 to +6) as described by van der Geest and Hall, 1996. Examples of promoters that are active in both the seed and non-seed tissue include 35S (Comai et al., 1992; Fraley et al., 1996) and 34S (Comai et al., 2000).

Seed-oil suppression genes which are examples of suitable transgenes for use in the second embodiment of the invention include, but are not limited to, sequences that direct cosuppression, antisense, immunomodulation, targeted ribozyme activity, transcription factor suppression or RNAi inhibition of the expression of one or several of the following target genes: carbonic anhydrase (CA) (Hoang and Chapman, 2002a), ACCase (Reverdatto et al., 1999), lysophosphatidic acid acyltransferase (LPAT) (Ohlrogge and Jaworski, 1997), diacyglycerol acyltransferase (DGAT) (Ohlrogge and Jaworski, 1997; Roesler et al., 1997), and oleosin expression (Sarmiento et al., 1997). In a preferred embodiment, the plant-active promoter is the seed-specific promoter from the cotton alpha-globulin gene (AGP) described by Sunilkumar et al., 2002 (the disclosures of which are hereby incorporated by reference), which is operably linked to a DNA sequence composed of a linear arrangement of partial sense and antisense sequences, expression of which construct would form RNA interference, representing genes that are both early in the seed-oil biosynthesis pathway, such as CA and ACCase genes, and late in the seed-oil biosynthesis pathway, such as LPAT and DGAT genes, in a synthetic construct. This construct is introduced into a cotton plant, using techniques known to those well versed in the art, to suppress seed-oil production. See Abbott et al., (2002) and Sunilkumar and Rathore (2001), the disclosures of which are hereby incorporated by reference. The suppression of seed-oil biosynthesis in plants, for example in cotton, generates pools of sucrose that are available for an increase in the production of commercially valuable cellulosic, starch or protein macromolecules such as cotton fibers, industrial starch, or pharmaceutical proteins.

The present invention involves, in a third embodiment, transgenic plants that contain a seed-oil suppression gene the expression of which can be controlled by application of an external stimulus. In accordance with the third embodiment of the invention, the plant contains a series of transgenic sequences that includes an exogenously inducible plant-active promoter linked to a seed-oil suppression gene. In the third embodiment, the expression of the seed-oil suppression gene occurs in the seed and/or other tissues when the external stimulus is present.

In this embodiment, the seed-oil suppression gene, as described above for the second embodiment of the invention, is operably linked to an inducible promoter that can respond to an external stimulus. Inducible promoters include, but are not limited to, the promoter from the ACE1 system which responds to copper (Mett et al., 1993); the promoter of the maize Intron 2 gene which responds to benzenesulfonamide herbicide safeners (Hershey et al., 1991 and Gatz et al., 1994), the promoter of the Tet repressor from Tn10 (Gatz et al., 1991), the phosphate-deficiency responsive promoter from a phosphate-starvation responsive beta-glucosidase gene from Arabidopsis (Lefebvre et al., 2001) and the synthetic promoter containing a 235bp sulfur deficiency response element from a soybean beta-conglycinin gene linked to a 35S core promoter sequence (Fujiwara et al., 2002). Inducible promoters that respond to an inducing agent to which plants do not normally respond are particularly useful, however any inducible plant-active promoter is contemplated for use in this invention. An exemplary preferred inducible promoter is the inducible promoter from a steroid hormone gene, the transcriptional activity of which is induced by a glucocorticosteroid hormone (Schena et al., 1991; the disclosures of which are hereby incorporated by reference) or the chimeric transcription activator, XVE, for use in an estrogen receptor-based inducible plant expression system activated by estradiol (Zuo et al., 2000; the disclosures of which are hereby incorporated by reference).

Other inducible promoters for use in plants are described in Ryals et al., 1989; Caddick et al., 1993 and Jepson, 1997, the disclosures of which are incorporated by reference. In a preferred embodiment, the ecdysone or estrogen receptor-based inducible plant expression system activated by insecticides with ecdysterodial activity or by estradiol (Martinez et al., 1999; Dhadialla et al., 1998; Unger et al., 2002; Zuo et al., 2000) is used to control the expression of the seed-oil suppression gene. The introduction of the ecdysone- or estrogen/receptor-based-systems promoter-driven seed-oil suppression gene into a plant allows propagation of transgenic plants that produce seed with normal seed-oil contents until the suppression of seed-oil content is required or preferred for commercial purposes.

When suppression of seed-oil content is desired, the transgenic plant or its developing seed can be treated with the chemical specific for the activation of the promoter system (e.g., non-steroidal ecydsteroid agonists or estradiol). Once the SOS gene becomes active, the synthesis or storage of seed-oil in the transgenic cotton plant is suppressed. In plants made according to this embodiment of the invention, the suppression of seed-oil synthesis or storage continues as long as the activating chemical is present in the cells of the plant. The suppression of seed-oil generates transient pools of sucrose in the resultant cottonseed that are available to stimulate production of commercially valuable cellulosic, starch or protein macromolecules such as cotton fibers, industrial starch, or pharmaceutical proteins. In this preferred embodiment, the seed-oil content is reduced to about 1% to 17% of the fuzzy whole seed weight.

A fourth embodiment of the present invention involves transgenic plants in which the controlled expression of an SOS gene is activated by application of an external stimulus, but also continues even in the absence of continued application of the external stimulus to maintain the expression. In accordance with the fourth embodiment, sequences introduced into a plant may include (1) a plant-active promoter linked to a seed-oil suppression gene but separated by a blocking sequence that is bounded on either side by specific excision sequences and (2) an inducible plant-active promoter, the function of which is sensitive to an external stimulus operably linked to a gene encoding a site-specific recombinase capable of recognizing the specific excision sequences. In a closely related embodiment, sequences introduced into a plant may include (1) a plant-active promoter linked to a seed-oil suppression gene but separated by a blocking sequence that is in turn bounded on either side by specific excision sequences; (2) a repressible plant-active promoter operably linked to a gene encoding a site-specific recombinase capable of recognizing the specific excision sequences and (3) a gene encoding a repressor specific for the repressible promoter the function of which is diminished by an external stimulus. In both of these types of embodiments of the invention, the seed-oil suppression gene is not expressed unless activated by excision of the blocking sequence by activation of a specific recombinase. The recombinase is not active unless the external stimulus is applied. Upon application of the stimulus, either the inducible promoter is activated or repressor function is inhibited, the recombinase is expressed and removes the blocking sequence at the specific excision sequences. The SOS gene becomes directly linked to the transiently-active promoter and is expressed.

As in the previous embodiment, by controlling the expression of genes that affect the seed-oil content of a plant, it is possible to grow plants under one set of conditions or in one environment where it is advantageous not to alter the oil content of the seed, then alter the environment directly, or indirectly by moving the plant or its seed to another set of conditions or another environment where the alteration of seed-oil content is advantageous. The transgenic plants of the fourth embodiment of this invention are prepared by introducing into their genome a series of functionally related DNA sequences containing the following basic elements: (1) a plant-active promoter, (2) a seed-oil suppression gene which is linked to the plant-active promoter with a blocking sequence separating the plant-active promoter and the gene, (3) unique specific excision sequences flanking the blocking sequence where the specific excision sequences are recognizable by a site-specific recombinase, (4) a gene encoding the site-specific recombinase, and (5) an inducible or repressible plant-active promoter capable of induction (or cessation of repression) through the application of an exogenous stimulus to either the seed or growing plant, linked to the recombinase gene. While these elements may be arranged in any order that achieves the interactions described below, in one embodiment they are advantageously arranged as follows: a DNA sequence contains the plant-active promoter, a first specific excision sequence, the blocking sequence, a second specific excision sequence, the seed-oil suppression gene and an inducible plant-active promoter operably linked to the site-specific recombinase gene that recognizes the specific excision sequences that are linked to each end of the blocking sequence.

When a plant contains the basic elements of the embodiment, the seed-oil suppression gene is not active. It is separated from its promoter by the blocking sequence. In the absence of the activating external stimulus, the inducible plant-active promoter is not active and the site-specific recombinase is not produced. Upon application of the activating external stimulus, the site-specific recombinase gene is transcribed, resulting in the production of the site-specific recombinase protein throughout the tissues of the transgenic plant. The site-specific recombinase recognizes the introduced specific excision sequences in the genome of the transgenic plant and physically removes the blocking DNA sequence situated between them. The removal of the blocking DNA sequence results in the operable linkage of the introduced plant-active promoter to the seed-oil suppression gene. The expression of the seed-oil suppression gene is activated when the plant-active promoter becomes active.

The majority of the genetic elements that constitute this embodiment have been described in the earlier embodiments. The recombinase/excision sequence system can be any one that selectively removes DNA in a plant genome. Excision sequences preferably are unique in the plant, so that unintended cleavage of the plant genome does not occur. Several examples of such systems are discussed in Sauer, 1990 and in Sadowski, 1993, the disclosures of which are hereby incorporated by reference. The bacteriophage CRE/LOX system, wherein the CRE protein performs site-specific recombination of DNA at LOX sites, is preferred. Other systems include, but are not limited to, the resolvases (Hall et al., 1993), FLP (Pan et al., 1993), SSV1 encoded integrase (Muskhekishvili et al., 1993), and the maize Ac/Ds transposon system (Shen and Hohn, 1992). Any method known to those skilled in the art is contemplated for use with this invention. See also the methods described by Oliver et al. in U.S. Pat. Nos. 5,723,765, 5,925,808 and 5,977,441.

In preferred methods and plants of this fourth embodiment, the plant-active promoter ultimately used to drive the expression of the seed-oil suppression gene is a seed-specific promoter, most preferably the cotton alpha-globulin promoter (AGP) described by Sunilkumar et al., 1997, the disclosures of which are hereby incorporated by reference. The seed-oil suppression gene preferably contains a linear arrangement of partial sense and antisense sequences representing genes that are both early in the seed-oil biosynthesis pathway, such as the CA and ACCase genes, and late in the seed-oil biosynthesis pathway, such as the LPAT and DGAT genes, as described above for the second embodiment of this invention, to create RNA interference. The inducible plant-active promoter used to drive the expression of the site-specific recombinase gene preferably is either the ecdysone (Martinez et al., 1999) or estrogen (Zuo et al., 2000) receptor-based inducible plant expression system activated by non-steroidal ecdysteroid agonists or estradiol, respectively, or derivatives thereof, as described above for the third embodiment of this invention. The preferred recombinase/excision sequence system is the bacteriophage CRE/LOX system, wherein the CRE protein performs site-specific recombination of DNA at LOX sites as described in Sauer, U.S. Pat. No. 4,959,317.

The preferred elements of this gene expression control system are advantageously arranged in the order described above and introduced into the genome of a cotton plant by methods well known to those versed in the art. The suppression of seed-oil biosynthesis is facilitated in the resulting transgenic plant only following the application of the chemical specific for the activation of the promoter system, for example a ecdysone- or estrogen-receptor-based promoter system. This results in transgenic plants that can produce seed with normal seed-oil content until the suppression of seed-oil content is required or preferred for commercial purposes and the promoter system is activated.

Once the seed-oil suppression gene becomes active as the result of the physical removal of the blocking sequence, the synthesis or storage of seed-oil in the transgenic cotton plant is suppressed for the life of the plant or seed irrespective of whether or not the activating chemical continues to be present. This generates stable pools of sucrose in the resultant plant that are available to increase, in a sustained fashion, the production of commercially valuable cellulosic, starch or protein macromolecules such as cotton fibers, industrial starch, or pharmaceutical proteins. The seed-oil content preferably is reduced to a level of 1% to 17% of the fuzzy whole seed weight. Permanent activation of the seed-oil suppression gene in the transgenic plant allows for the commercial use of the plant or seed without continued application of the activating chemical. This attribute of the embodiment is particularly advantageous for agricultural and horticultural practices.

The present invention involves, in a fifth embodiment, transgenic parental plants that are hybridized to produce a progeny plant expressing a seed-oil suppression gene not expressed in either parent. Methods of the fifth embodiment do not employ a repressor gene or an inducible or repressible promoter. Instead, a recombinase transgene is linked to a second plant-active promoter and introduced into a separate, second plant. The first plant, which contains a plant-active promoter and an SOS gene, blocked as described above with a blocking sequence bounded by specific excision sequences, is hybridized with the second plant containing the recombinase gene, producing progeny that contain all of the sequences necessary for expression of the SOS gene. When the second plant-active promoter becomes active, the recombinase removes the blocking sequence in the progeny, allowing expression of the seed-oil suppression gene in the progeny when it was not expressed in either parent.

The fifth embodiment of the present invention is a modification of the fourth embodiment in which the control of the seed-oil suppression gene is effected by the hybridization of two transgenic parent plants, neither of which express the seed-oil suppression gene. In this embodiment, a transgenic parent plant that contains the following genetic operably linked elements: a first plant-active promoter, a blocking sequence flanked on either side by specific excision sequences, and a seed-oil suppression gene, is crossed to a separate, second parent transgenic plant that contains the following operably linked elements: a second plant-active promoter and a site-specific recombinase gene.

The progeny from this hybridization contain all of the genetic elements necessary for expression of the SOS gene, contained in both of the parental plants combined. In the progeny, upon activation of the second plant-active promoter, the site-specific recombinase gene is transcribed and the recombinase protein is produced. This causes the excision of the blocking sequence and the activation of the seed-oil suppression gene under the control of the first plant active promoter. The progeny of the hybridization cross thus express the seed-oil suppression phenotype (a trait not expressed in either parent).

Preferably, the first plant-active promoter (that is used ultimately to drive the expression of the seed-oil suppression gene and is contained in the first transgenic parental plant) is a seed-specific promoter, most preferably the cotton alpha-lobulin promoter (AGP) described by Sunilkumar et al., 1997. The seed-oil suppression gene preferably is composed of an RNAi sequence for genes that are both early in the seed-oil biosynthesis pathway, such as the CA and ACCase genes, and late in the seed-oil biosynthesis pathway, such as the LPAT and DGAT genes, as described above for the second embodiment of this invention. The specific excision sequences flanking the blocking sequence physically separating the first plant-active promoter and the seed-oil suppression gene preferably are those of the CRE/LOX system described by Sauer, U.S. Pat. No. 4,959,317.

Preferably, the second plant-active promoter (that is used to drive the expression of the site-specific recombinase gene and contained in the second transgenic parental plant) is a germination-specific promoter or other tissue-specific promoter that is not expressed in the developing embryo. The preferred recombinase gene is the bacteriophage CRE gene described in Sauer, U.S. Pat. No. 4,959,317. The preferred elements of this embodiment advantageously are arranged in the order described above and introduced into the genome of a cotton plant by methods well known to those versed in the art. The suppression of seed-oil biosynthesis in a transgenic cotton plant is facilitated only in the progeny resulting from the hybridization of the two transgenic parental lines as described. This allows for the propagation of the transgenic parental plants with normal seed-oil content, to produce transgenic hybrid seed of normal seed-oil content that will generate progeny plants that exhibit the suppressed seed-oil content phenotype. Once the seed-oil suppression gene becomes active in the progeny as a result of the physical removal of the blocking sequence, the synthesis or storage of seed-oil in the transgenic cotton plant will be suppressed for the life of the plant or its progeny. This generates stable pools of sucrose in the resultant cottonseed that are available to increase, in a sustained fashion, the production of commercially valuable cellulosic, starch or protein macromolecules such as cotton fibers, industrial starch, or pharmaceutical proteins. Preferably, the seed-oil content is reduced to a level of 1% to 17% of the fuzzy whole seed weight. The ability to sell seed of normal seed-oil content that will produce progeny expressing the seed-oil suppression phenotype is particularly advantageous for agricultural and horticultural purposes.

The sixth embodiment of the present invention is a modification of the fifth embodiment. In this embodiment, the expression of an SOS transgene in the transgenic plant is controlled by expression of a second gene introduced into the plant by direct transfection with exogenous DNA. Therefore, in accordance with this sixth embodiment, the sequences encoding the site-specific recombinase are introduced separately into a transgenic plant that contains the blocked seed-oil suppression gene, via direct transfection using a viral vector or any other method known in the art.

In the sixth embodiment, transgenic plants are generated that contain the following genetic operably linked elements: a plant-active promoter, a blocking sequence flanked on either side by specific excision sequences and a seed-oil suppression gene. The seed-oil suppression gene in this transgenic plant is inactive as a result of the physical separation of the gene from its plant active promoter. The site-specific recombinase gene required to effect excision of the blocking sequence, thus generating an active seed-oil suppression gene (under control of the plant-active promoter), is introduced into the transgenic plant containing the blocked seed-oil suppression gene construct by transfection with a viral vector containing the site- specific recombinase gene or by any convenient method known in the art. The site-specific recombinase gene preferably is delivered as part of an infectious RNA viral vector for direct translation into an active protein (see Lawrence and Novak, (2001), the disclosures of which are hereby incorporated by reference), in an infectious cDNA viral vector for transcription and translation into an active recombinase protein (Baulcombe et al., 2001) or by any convenient known method.

In this embodiment, transgenic plants containing the blocked seed-oil suppression gene rapidly generate high levels of the recombinase protein throughout the plant following infection with the engineered viral vector containing the site-specific recombinase. Expression of the site-specific recombinase results in excision of the blocking sequence and the activation of the seed-oil suppression gene under the control of the first plant active promoter. The infected plants therefore express the seed-oil suppression phenotype (a trait not expressed in the uninfected parent). Once the seed-oil suppression gene becomes active in the infected plants as the result of the physical removal of the blocking sequence, the synthesis or storage of seed-oil in the transgenic plant is suppressed for the life of the plant or its progeny. This generates stable pools of sucrose in the resultant seed that are available to increase, in a sustained fashion, the production of commercially valuable cellulosic, starch or protein macromolecules such as cellulose fibers, industrial starch, or pharmaceutical proteins. Preferably, the seed-oil content is reduced to a level of 1% to 17% of the fuzzy whole seed weight. The ability to sell seed of normal seed-oil content that will produce plants that cannot express the seed-oil suppression phenotype until treated with the engineered viral vector is particularly advantageous for agricultural and horticultural purposes.

In the fourth, fifth and sixth embodiments, the seed-oil suppressing gene is expressed when the plant-active promoter operably linked to it becomes active, and will continue to be expressed so long as the plant-active promoter is active, without continuous external stimulation. These systems are particularly useful for developing seed where a particular trait is only desired during the first generation of plants grown from that seed, or a trait is desired only in subsequent generations.

In a seventh embodiment, the invention relates to a method of breeding plants to optimize the effects of a yield enhancement transgene such as an SOS gene. In this method, a donor parent containing the transgenic yield enhancing gene and also contains a unique phenotype or set of phenotypes (such as dwarfing, large bolls and precocious flowering, for example) that contribute multiple phenotypic traits is crossed with elite recurrent parents that previously had been selected for yield, fiber quality and/or agronomic performance. Progeny of this cross can be selected for an optimal combination of traits which allow the enhanced yield transgene to produce the best results. The donor parent may be used for limited backcrossing with elite recurrent parents, which transfers the yield-enhancing transgenetic trait along with a subset of the multiple phenotypic traits from the donor parent into the elite recurrent parent germplasm. Subsequent selection of optimal combinations allow maximum performance of the yield enhancing trait. Limited backcrossing is performed to ensure that the derived lines contain the genetic compliment of the elite recurrent parent, the yield-enhancing transgene and at least some of the potentially beneficial enhancing phenotypic traits contributed by the donor parent. These can be selected to identify the most beneficial combination of traits for each transgenic plant line.

The invention is further illustrated by the following examples.

### EXAMPLES

### Example 1.

### Seed-oil Content is Highly Correlated to Fiber Yield

Twenty-one different cotton varieties over a period of twelve years were compared by the USDA-ARS to determine the relationship between various seed parameters and lint yield (Rayburn et al., 1989 through 2001). The strong influence of environment on cottonseed oil content requires multi-site data sets and standardized protocols to identify significant relationships. Only varieties that were present in 7 or more region-years were included in the comparison. Only the non-Acala picker cotton regions were included: Eastern, Delta and Central. The high fiber quality check variety, Acala Maxxa (bred in California) was excluded from the comparison due its poor adaptability in these three regions (17% lower average lint yield than the next lowest yielding variety). Each region-year mean observation was derived from 3 to 5, four-replication trial sites. General Linear Model Least Square Means (SAS, 2002) were determined from a minimum of 21 sites and 84 observations to reduce environmental influence on seed parameters. The mean variety parameters then were regressed against each other to derive the following table of correlation coefficients and the following regression equations.

TABLE 1.1
Simple Correlation Coefficients and Regression Equations
between Measured Parameter Means for 21 Cotton Varieties (r).

| | Seed | | Oil | |
|---|---|---|---|---|
| | Index | Nitrogen | Percent | Lint |
| | (g/1.00 seed) | Percent | (fuzzy seed) | Percent |
| Lint Yield | −0.5751** | −0.2997 | −0.7671** | 0.8155** |
| (lbs/acre | | | | |
| Lint Percent | −0.4206 | 0.1044 | −0.7401** | |
| Oil Percent | 0.5577** | 0.2919 | | |
| Nitrogen | 0.4382* | | | |
| Percent | | | | |

Equation 1.1: Lint Yield (lbs/acre) = 2619 − 81.87 × Seed-oil % (R² = 0.5884).
Equation 1.2: Lint Yield = 668 + 46.94 × Lint % − 428 × Seed N % (R² = 0.8149).
Equation 1.3: Lint Yield = 2664 − 69.14 × Seed-oil % − 29.96 × Seed Index (R² = 0.6199).
Correlations are designated significant at P = 0.05 (*) or significant at P = 0.01 (**). (Rayburn, 1989-2001).

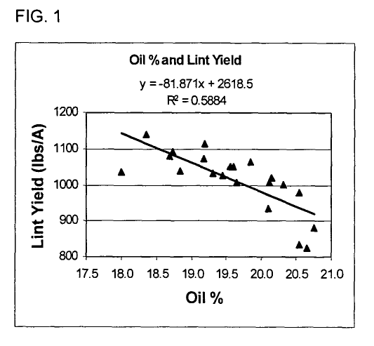

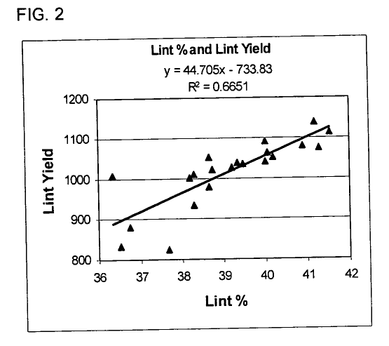

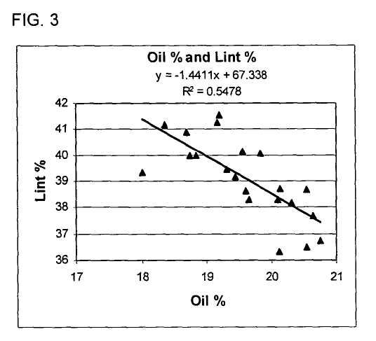

Lint yield was most related to lint percent, r=0.8155. See FIG. 2. Lint percent has been well recognized as a useful parameter for selection when pursuing higher lint yield. Oil percent was highly negatively correlated with lint yield, r=−0.7671 (see FIG. 1), despite positive plant breeding selection for both lint yield and oil percent (Cherry et al., 1986). Lint percent and oil percent are also highly negatively correlated. See FIG. 3. Lint percent is less subject to environmental control because it is calculated from two parameters which are each influenced by environment in a similar manner: fiber mass and fiber+seed mass. Oil percent is highly impacted by environment, therefore it is difficult to determine whether the impact of lint percent on yield is direct or derives indirectly from the impact of oil percent on fiber mass.

When all parameters were included (see Equation 1.2), stepwise multiple regression identified lint percent and N (Nitrogen) percent as significant contributors to lint yield. When lint percent was excluded, oil percent was most significantly related to lint yield, followed by seed index. In Equation 1.1, the regression of oil percent to lint yield, each percent reduction in seed-oil percent increased lint yield by 82 lbs per acre; an 8.2% increase in yield at the mean yield of 999 lbs per acre for these 21 varieties. Using Equation 1.1, the mean lint yield of 999 lbs per acre and the mean lint percent of 39%, every pound per acre reduction in seed-oil results in an increase in lint of 5.3 pounds per acre. This increase ratio is consistent with the greater energy cost to produce a pound of oil versus a pound of cellulose in the seed.

Considering that these varieties were selected for high lint yield and high lint %, and not for low oil percent, by 15 different breeders, the significant relationship between oil percent and lint yield provides a strong example of the lint yield enhancement that can be achieved from genetic manipulation that deliberately lowers seed-oil content below that which is available within elite germplasm currently.

### Example 2

### DP 555 BG/RR and DP 493 Reduced Seed-oil Varieties with Enhanced Yield

Cotton varieties bred by Delta and Pine Land Company, DP 555 BG/RR and DP 493, exhibit significant yield improvement and novel growth pattern compared with other varieties available in the market. DP 555 BG/RR and DP 493 were compared with 8 elite varieties with similar maturities. Lint yields of these elite cultivars ranged from 77 to 102 percent of DP 555 BG/RR. Between 29 and 143 trial sites (N) were included in each comparison. DP 555 BG/RR and DP 493 sustain new node and fruiting site production longer during the boll loading period than other varieties. For this reason, early season growth regulators often are required to avoid excess plant height, a plant morphological trait consistent with increased sucrose supply. Using the AOCS Aa 4-38 cotton seed-oil analytical procedures (AOCS, 2001) oil content was determined for this set of 10 varieties, grown in three replicated trial sites. Samples were first acid delinted to remove the fuzz fibers (linters).

DP 555 BG/RR and DP 493 exhibit both significant yield enhancement and low oil content. The relationship between lint yield and oil percent derived in Example 1, Equation 1. 1, was used to calculate an expected yield based on the oil percent. Observed and expected lint yields were compared using Chi-square analysis and not found to be significantly different. The variety with the largest deviation between observed and expected was DP 5415RR. The non-transgenic version of this variety, DP 5415, also had the largest deviation between observed and expected in the USDA data set from 1989 through 2001 (which did not include DP 555 BG/RR, see FIG. 1, data point with lowest oil content).

TABLE 2.1
Measured parameters from 10 elite varieties and
predicted lint yield using equation 1.1 above.

| | | | | | Expected |
|---|---|---|---|---|---|
| | Obs. Lint | | Oil | | Yield |
| | Yield | | Percent | Seed Index | (from |
| | (% of | Sites | (delinted | (g/100 | equation |
| Variety | DP555) | (N) | seed) | seeds) | 1.1) |
| DP 493 | 102 | 48 | 15.1 | 7.9 | 105 |
| DP 555 BR | 100 | na | 16.0 | 9.0 | 100 |
| DP 491 | 91 | 110 | 17.9 | 9.4 | 88 |
| NuCOTN 33B | 87 | 117 | 19.1 | 10.0 | 81 |
| DP 565 | 87 | 118 | 16.9 | 9.6 | 94 |
| FM 966 | 87 | 29 | 18.4 | 12.4 | 85 |
| FM 989 BR | 85 | 80 | 18.2 | 12.6 | 86 |
| DP 5415 R | 83 | 143 | 17.5 | 8.90 | 91 |
| ST 4793 R | 81 | 136 | 18.5 | 12.0 | 84 |
| FM 989 R | 77 | 122 | 17.2 | 11.4 | 93 |

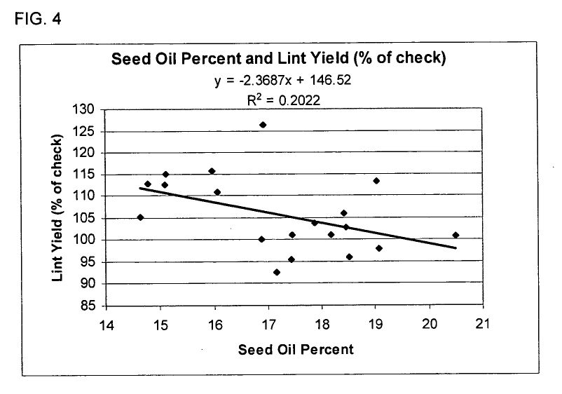

When both commercial varieties and experimental lines were included in the analysis of the relationship between fiber yield and seed parameters, statistically significant relationships were developed between fiber yield (as a percent of a common conventional check, DP 565) and seed-oil percent. FIG. 4 displays this relationship for the above elite varieties and 9 experimental lines. Even though seed-oil percent was not used as a selection criteria, several of these modern high yielding lines and cultivars from diverse germplasm sources demonstrated low seed-oil percentages.

The low seed-oil content of DP 555 BG/RR and DP 493 reduces the metabolic cost to mature bolls, thus providing greater carbohydrate resources for new node and fruiting site production, sustained boll retention and fiber maturation. Although DP 555 BG/RR and DP 493 were not selected for low seed-oil content, its growth habit and lint yield is consistent with the equations derived from the evaluation of 21 diverse varieties evaluated over a 12-year period in Example 1.

### Example 3

### Inheritance of Low Seed-Oil in Cotton

Multiple transgenic lines with the same recurrent parent as DP 555 BG/RR were analyzed for seed-oil content using the AOCS Aa 4-38 analytical procedures (AOCS, 2001). The following results were obtained.

TABLE 3.1
Measured seed-oil percent from delinted seed of 9 lines
that were derived from the recurrent parent of DP 555 BG/RR.

| | Oil Percent |
|---|---|
| DP 555 BG/RR line | (delinted seed) |
| Line 1 | 17.8 |
| Line 2 | 18.2 |
| Line 3 | 17.5 |
| Line 4 | 16.8 |
| Line 5 | 17.6 |
| Line 6 | 16.5 |
| Line 8 | 16.2 |
| Line 9 | 19.0 |
| Line 10 | 17.8 |

Thirty-three percent (3 out of 9) of the lines express a low seed-oil phenotype (lines 4, 6 and 8). This is consistent with the hypothesis that the inheritance of the DP 555 BG/RR reduced seed-oil phenotype is a recessive allele single gene trait, however further genetic inheritance studies will be required to fully test this hypothesis. Out of a segregating population derived from a heterozygous plant for a recessive allele, we expect 25% of the lines to express the recessive phenotype (homozygous recessive) and 75% of the lines not to express the recessive phenotype (25% homozygous for the dominant allele and 50% heterozygous for the locus). Since recessive alleles often result from a change in the DNA sequence of either regulatory elements or enzyme coding regions, the existence of a recessive allele that confers the reduced seed-oil phenotype is consistent with the objective of this invention—to identify and create heritable alleles and transgenes that impair the normal biosynthesis and storage of seed-oil, thereby increasing the sucrose supply for sustained vegetative growth, boll retention and fiber maturation.

### Example 4

### Inducible Gene with Single Application of the Activation Activating Agent

A site-specific recombinase driven gene rearrangement combined with a chemically inducible or repressible promoter system permits activation of the yield enhancing gene without the need for continuous application of the exogenous activating agent. Chemical activation or derepression of a site specific recombinase gene causes a blocking sequence to be physically removed. This allows the desired target gene to be controlled by a suitable developmentally controlled promoter. Once the chemical activation or derepression of the site-specific recombinase is achieved, the continued presence of the external chemical agent is not required to maintain derepression. See Oliver and Velton (2001).

The known genetic system for controlling gene expression described in Oliver et al. (U.S. Pat. Nos. 5,723,765; 5,925,808 and 5,977,441) and Oliver and Velten (2001), the disclosures of which are hereby incorporated by reference, are suitable for use in transgenic plants of this invention. These methods therefore can be used conveniently to express the yield enhancing trait (a transgene such as a seed-oil suppression transgene) and with the invention. A single exposure to an external stimulus (for example the antibiotic tetracycline) results in the derepression of a CRE recombinase gene. The CRE recombinase is expressed and directs removal of a blocking sequence flanked by appropriate excision sequences to generate a permanent genetic rearrangement. In this new DNA rearrangement, a germination disruption gene, for example a ribosomal inhibitory protein or the endonuclease Barnase (both of which disrupt protein synthesis), is under the control of a suitable promoter such as the Late Embryogenesis Abundant (LEA) promoter. Because the new genetic arrangement is permanent, the gene becomes activated and stays activated, even without the continuing presence of tetracycline, the initial activating external agent in this example.

Genetic constructs are developed using the methods described in U.S. Pat. No. 5,723,765. The genetic constructs included a modified 35S CaMV promoter containing three tandemly arranged synthetic tet operon sequences, positioned as described by Gatz et al. (1988), the disclosures of which are hereby incorporated by reference, operably linked to a CRE recombinase coding sequence. This gene construct is referred to as 35SopCRE. In addition, a cotton LEA4 promoter fragment (a genomic fragment 5′ to the transcriptional start site of the LEA gene of cotton linked 3′ to a blocking sequence, containing a tet repressor gene in reverse orientation driven by a 35S CaMV promoter and completely flanked by artificially constructed mutant LOX sites) is linked 5′ to a plant ribosomal inhibitory protein (RIP) coding sequence. A gene construct as described above but having the RIP sequence replaced with a Barnase coding sequence isolated by PCR from the genome of Bacillus amyloliquefaciens also is produced. Each gene construct is introduced individually into a virulent strain of Agrobacterium tumefaciens by direct transformation. The constructs then are introduced, via Agrobacterium infection, into the plant, for example tobacco (Nicotiana tobacum). The presence of the individual genetic components within the genomes of the primary transformants may be confirmed by standard PCR analysis.

The efficacy of the tet repression of the 35Sop promoter and the efficiency of derepression by exposure to tetracycline are verified by germination of R1-F1 seed on solid media containing various concentrations of tetracycline ranging from zero (for analysis of repression) to 100 μg/ml. Analysis of CRE recombinase activity under these conditions is based on the presence or absence of the blocking sequence contained within the flanking LOX sites (the target sequences for the CRE recombinase). In this scheme, the blocking sequence contains the tet repressor gene, which can be assayed by northern analysis. Northern analyses of seedlings derived from seeds treated with varying levels of tetracycline indicate that at between 50 and 100 μg/mL tetracycline, excision of the tet repressor blocking sequence occurred in the majority of the cells of the seedlings in this system. RNA samples derived from individual whole seedlings do not exhibit any detectable accumulation of tet repressor transcript. These data indicate that the tet repressor/tet promoter system and CRE/LOX recombination is functional and highly effective, demonstrating the effectiveness of this particular method.

In preferred embodiments, the recombinase gene is under the control of a promoter that is directly and tightly regulated by the application of an external stimulus. In addition, a seed specific promoter that is chosen to impact lipid biosynthesis during normal seed maturation more directly replaces the LEA promoter. For seed-oil suppression, the germination disruption component is replaced with a seed-oil suppression gene-coding region. Thus, in a preferred embodiment, an external stimulus would be applied to the transgenic plant (e.g. cotton) seed to induce the expression of the recombinase which removes a blocking sequence to generate a seed-oil suppression gene that is active only during development of next generation seeds.

References

### U.S. PATENT DOCUMENTS

1. 4,771,002, Gelvin.

2. 4,959,317, Sauer.

3. 5,102,796, Hall et al.

4. 5,106,739, Comai et al

5. 5,182,200, Slightom et al.

6. 5,378,619, Rogers.

7. 5,428,147, Barker et a.

8. 5,492,820, Sonnewald.

9. 5,530,196, Fraley et a.

10. 5,563,328, Mitra et al.

11. 5,716,837, Barry et al.

12. 5,723,765, Oliver et al.

13. 5,739,082, Donn.

14. 5,795,753, Cigan et al.

15. 5,850,019, Maiti et al.

16. 5,856,177, Grula et al.

17. 5,917,127, Willmitzer et al.

18. 5,925,808, Oliver et al.

19. 5,945,579, Smith.

20. 5,955,651, Coruzzi et al.

21. 5,959,187, Bailey et al.

22. 5,977,441, Oliver et al.

23. 5,981,852, Van Assche et al.

24. 5,981,836, Osteryoung

25. 5,986,173, Smeekens et al.

26. 5,998,700, Lightfoot et al.

27. 6,025,542, Smeekens et al.

28. 6,051,753, Comai et al.

29. 6,057,493, Willmitzer et al.

30. 6,107,547, Coruzzi et al.

31. 6,143,950, Chory et al.

32. 6,156,954, Zhong et al.

33. 6,166,293, Doerner et al.

34. 6,175,060, Lefebvre et al.

35. 6,184,440, Shoseyov et al.

36. 6,222,098, Barry et al.

37. 6,235,971, Barry et al.

38. 6,245,967, Sonnewald et al.

39. 6,245,969, Chory et al.

40. 6,252,139, Doerner et al.

41. 6,352,846, Chory et al.

42. 6,420,629, Xue et al.

43. 6,423,885, Waterhouse et al.

44. 6,441,277, Barry et al.

45. 6,444,876, Lassner et al.

46. 6,462,257, Perera et al.

47. 6,465,718, Inze et al.

48. 6,476,294, Lassner et al.

49. 6,476,295, Barry et al.

50. 20020023282, Gaxiola et al.

51. 20020066121, Kosegi et al.

52. 20020069430, Kisaka et al.

53. 20020069431, Broadway et al.

54. 20020138875, Barry et al.

55. 20020170091, Lassner et al.

### FOREIGN PATENT DOCUMENTS

1. EP 0332104, Ryals et al.

2. EP 0608359, Kerr.

3. WO 93/07742, Kerr.

4. WO 93/21334, Caddick et al.

5. WO 97/06269, Jepson.

6. WO 97/39112, Chory et al.

7. WO 97/44471, Kossmann et al.

8. WO 98/03631, Doerner et al.

9. WO 98/11240, Ellis.

10. WO 98/12913, Bailey et al.

11. WO 98/58069, Barry et al.

12. WO 98/59039, Chory et al.

13. WO 99/58654, Neuhaus et al.

14. WO 00/04761, Zhong et al.

15. WO 00/09724, Sheen et al.

16. WO 00/32760, Doerner et al.

17. WO 00/56905, De Veyider et al.

18. WO 00/63401, Habben et al.

19. WO 00/69883, Roberts et al.

20. WO 00/70062, Staub et al.

21. WO 00/73422, Quanz.

22. WO 00178984, Tomes et al.

23. WO 01/17333, Haigler et al.

24. WO 01/23594, Sun et al.

25. WO 01/64928, Giroux et al.

26. WO 01/66777, Eriksson et al.

27. WO 01170987, Jackson et al.

28. WO 01/90343, Martienssen et al.

29. WO 01/96579, Miskolczi et al.

30. WO 01/96580, Schmulling et al.

31. WO 02/16558, Gaxiola.

32. WO 02/18538, Kim.

33. WO 02/053589, De Veyider.

34. WO 02/61042, Liljegren et al.

35. WO 02/97101, Regierer et al.

### NON-PATENT PUBLICATIONS

1. Abbott JC, Barakate A, Pincon G, Legrand M, Lapierre C, Mila I, Shuch W, Halpin C (2002). “Simultaneous suppression of multiple genes by single transgenes. Downregulation of three unrelated lignin biosynthetic genes in tobacco.” Plant Physiol. 128:844-853.

2. American Oil Chemists' Society (2001). AOCS Official Method Aa 4-38, Cottonseed Oil. http://www.aocs.org

3. Atanassova R, Chaubet N, Gigot C (1992). “A 126 bp fragment of a plant histone gene promoter confers preferential expression in meristems of transgenic Arabidopsis.” Plant. 2:291-300.

4. Auld DL, Ethridge MD, Dever, JK, Dotray PD (1998) “Chemical mutagenesis as a tool in cotton improvement.” Proceedings: Beltwide Cotton Conf. 1:550-552.

5. Austin-Brown SL, Chapman KD (2002) “Inhibition of phospholipase Dα by N-acylethanolamines.” Plant Physiol. 129:1892-1898.

6. Bao X, Ohlrogge J (1999). “Supply of fatty acids is one limiting factor in the accumulation of triacylglycerol in developing embryos.” Plant Physiol. 120:1057-1062.

7. Bao X, Shorrosh BS, Ohirogge JB (1997). “Isolation and characterization of an Arabidopsis biotin carbyoylase gene and its promoter.” Plant Mol. Biol. 35:539-550.

8. Barker SJ, Harada JJ, Goldberg RB (1988). “Cellular localization of soybean storage protein mRNA in transformed tobacco seeds.” Proc. Natl. Acad. Sci. USA 85:458462.

9. Bassett DM, Kerby TA (1996). “A History of California Cotton.” In: Cotton Production Manual. Hake, Kerby and Hake, Eds. Univ of Calif., Oakland, pp. 111-122.

10. Baulcombe et al., 2001. Plant J. 25:237-245.

11. Bouvier-Nave P, Benveniste P. Oelkers P, Sturley SL, Schaller H (2002) “Expression in yeast and tobacco of plant cDNA encoding acyl CoA:diacylglycerol acyltransferase.” Eur. J. Biochem. 267:85-96.

12. Brown AP, Slabas AR, Denton H (2002) “Substrate selectivity of plant and microbial lysophasphatidic acid acyltransferases.” Phytochem. 61:493-501.

13. Brubaker CL, Bourland FM, Wendel JF (1999) “The Origin and Domestication of Cotton.” In: Cotton: Origin, History, Technology and Production. Smith and Cothren, eds. John Wiley & Sons, Inc.

14. Burton JW (1987) “Quantitative Genetics: Results Relevant to Soybean Breeding.” In: Soybeans: Improvement, Production and Uses. Wilcox, ed. Am. Soc. Agron., Madison Wis. pps. 211-247.

15. Chapman KD, Austin-Brown S, Sparace SA, Kinney AJ, Ripp KV, Pirtle IL, Pirtle RM (2001) “Transgenic Cotton Plants with Increased Seed Oleic Acid Content.” J. Am. Oil Chemists Soc. 78:941-947.

16. Chapman KD, Moore TS Jr (1993) “Catalytic properties of a newly discovered acyletransferase that synthesizes N-acylphosphatidylethanolamine ” In: Cottonseed (Gossypium hirsutum L.) Microsomes. Plant Physiol. 102:761-769.

17. Chapman KD, Venables B, Markovic R, Blair RW Jr, Bettinger C (1999) N-acylethanolamines in seeds. Quantification of molecular species and their degradation upon imbibition.” Plant Physiol. 120:1157-1164.

18. Chen Z-L, Pan N-S, Beachy RN (1988) “A DNA sequence element that confers seed-specific enhancement to a constitutive promoter.: EMBO. J. 7:297-302.

19. Cherry JP, Kohel RJ, Jones LA, Powell WH (1986) “Food and Feeding Quality of Cottonseed.” In: Cotton Physiology. Chapter 37. Published by The Cotton Foundation.

20. Chuang CF, Meyerowitz EM (2000) “Specific and heritable genetic interference by double-stranded RNA in Arabidopsis thaliana.” Proc. Natl. Acad. Sci. USA 97:4985-4990.

21. Cornelissen M, Vandewiele M (1989) “Both RNA level and translocation efficiency are reduced by anti-sense RNA in transgenic tobacco.” Nucl. Acids Res. 17:833-843.

22. Cothren JT (1999) “Physiology of the Cotton Plant.” In: Cotton: Origin, History, Technology, and Production. Smith and Cothren, eds. John Wiley and Sons, Inc.

23. Cotten M, Biernstiel ML (1989) “Ribozyme mediated destruction of RNA in vivo.” EMBO. J. 8:3861-3866.

24. Dani RG (1999) “Genetic improvement of seed oil content, following indirect selection for earliness and fiber yield in cotton (Gossypium hirsutum L.).” Adv. Plant Sci. 12:479-492.

25. Dehesh K, Tai H, Edwards, Byrne J, Jaworski J (2001) “Overexpression of 3-ketoacyl-acyl-carrier protein synthase ills in plants reduces the rate of lipid synthesis.” Plant Physiol. 125:1103-1114.

26. Delmer DP (1999) “Cellulose biosynthesis: exciting times for a difficult field of study.” Annu. Rev. Plant Physiol. Plant Mol. Biol. 50:245-272.

27. Dhadialla TS, Carlson GR, Le DR (1998) “New insecticides with ecdysteroidal and juvenile hormone activity.” Annu. Rev. Entomol. 43:545-569.

28. Dudley J, Lambert RJ (1992) “Ninety generations of selection for oil and protein in maize.” Maydica 37:81-87.

29. Dudley J, Lambert RJ, de la Rocha IA (1977) “Genetic analysis of crosses among corn strains divergently selected for percent oil and protein.” Crop Sci. 17:111-117.

30. Dunwell JM (2000) “Transgenic approaches to crop improvement.” J. Exp. Bot. 51:487-496.

31. Ezcurra I, Ellerstrom M, Wycliffe P, Stalberg K, Rask K (1999) “Interaction between composite elements in the napA promoter: both the B-box ABA responsive complex and the RY/G complex are necessary for seed-specific expression.” Plant Mol. Biol. 40:699-709.

32. Eastmond PT, Germain V, Lange PR, Bryce JH, Smith SM, Graham IA (2000) “Postgerminative growth and lipid catabolism in oilseeds lacking the glyoxylate cycle.” Proc. Natl. Acad. Sci. USA 97:5669-5674.

33. Evans LS (1990) “Relationship between carbon dioxide enrichment and reproductive yield.” Proceedings: Beltwide Cotton Production Res. Conf., p. 716.

34. Fire A, Xu S, Montgomery MK, Kostas SA, Driver SE, Mello CC (1998) “Potent and specific genetic interference by double-stranded RNA in Caenorphabditis elegans.” Nature 291:806-811.

35. Focks N, Benning C (1998) “wrinkled 1: a novel, low-seed-oil mutant of Arabidopsis with a deficiency n the seed-specific regulation of carbohydrate metabolism. Plant Physiol. 118:91-101.

36. Fujiwara T, Awazuhara M, Chino M, Goto DB, Hayashi H, Kim H, Kim S-G, Matsui A, Naitob S (2002) “A 235-bp region from a nutritionally regulated soybean seed-specific gene promoter can confer its sulfur and nitrogen response to a constitutive promoter in aerial tissues of Arabidopsis thaliana.” Plant Sci. 163:75-82.

37. Galau GA (1986) “Differential gene activity in cotton embryogenesis.” In: Cotton Physiology. Chapter 28. Published by The Cotton Foundation.

38. Gatz C. et al., (1988) “TN 10-Encoded Tet Repressor Can Regulate An Operator-Containing Plant Promoter.” Proc. Natl. Acad. Sci. USA 85:1394-1397.

39. Gatz et al., (1991) Mol. Gen. Genet. 227:229-237.

40. Gatz et al., (1994) Mol. Gen. Genet. 243:32-38.

41. Greenplate J, Penn S, Dahm A, Reich B, Osborn J, Mullins W (2002) “Boligard II: dual toxin expression and interaction.” Proceedings: Beltwide Cotton Conf. National Cotton Council, Memphis Tenn.

42. Guan X, StegeJ, Kim M, Dahmani Z, Fan N, Heifetz, P, Barbas CFIII, Briggs SP (2002) “Heritable endogenous gene regulation in plants with designed polydactyl zinc finger transcription factors.” Proc. Natl. Acad. Sci. USA 99:13296-13301.

43. Hall, SC, Halford SE (1993) “Specificity of DNA recognition in the nucleoprotein complex for site-specific recombination by Tn21 resolvase.” Nucl. Acids Res. 21:5712-5719.

44. Hayaski M, Toriyama K, Kondo M, Nishimura M (1998) “2,4-Dichlorophenoxybutyric acid-resistant mutants of Arabidopsis have defects in glyoxysomal fatty acid b-oxidation.” Plant Cell 10: 183-195.

45. Hershey et al., (1991) Mol. Gen. Genet. 227:229-237.

46. Hoang CV, Chapman KD (2002a) “Biochemical and molecular inhibition of plastidial carbonic anhydrase reduces the incorporation of acetate into lipids in cotton embryos and tobacco cell suspensions and leaves.” Plant Physiol. 128:1417-1427.

47. Hoang CV, Chapman KD (2002b) “Regulation of carbonic anhydrase gene expression in cotyledons of cotton (Gossypium hirsutum L.) seedlings during post-germinative growth.” Plant Mol. Biol. 49:449458.

48. Hobbs DH, Lu C, Hills MJ (1999) “Cloning of a cDNA encoding diacylglycerol acyltransferase from Arabidopsis thaliana and its functional expression.” FEBS Lett. 452:145-149.

49. Hood EE, Helmer GL, Fraley RT, Chilton MD (1986) “The hypervirulence of Agrobacterium tumefaciens A281 is encoded in a region of pTiBo542 outside of TDNA.” J. Bactelol. 168:1291-1301.

50. Hopper NW, McDaniel (1999) “The Cotton Seed.” In Cotton: Origin, History, Technology and Production. Smith and Cothren, eds. John Wiley & Sons, Inc.

51. Horsch RB, Fry JE, Hoffman NL, Eicholtz D, Rogers SG, Fraley RT (1985) “A simple and general method for transferring genes into plants.” Science 227:1229-1231.

52. Ishimura K, Ohkawa Y, Ishige T, Tobias DJ, Ohsugi R (1998) “Elevated pyruvate orthophosphate dikinase (PPDK) activity alters carbon metabolism in a C₃ transgenic potatoes with a C₄ maize PPDK gene.” Physiologia Plantarum 103:340-346.

53. Jako C, Kumar A, Wei Y, Zou J, Barton DL, Giblin EM, Covello PS, Taylor DC (2001) “Seed-specific over-expression of an Arabidopsis cDNA encoding a diacylglycerol acyltransferase enhances seed oil content and seed weight.” Plant Physiol. 126:861-874.

54. Jobling SA, Jarmon C, The M-M, Holmberg N, Blake C, Verhoeyen ME (2002) “Immunomodulation of enzyme function in plants single-domain antibody fragments.” Nat. Biotech. 21:77-80.

55. Jones LA, King CC (1993) “Cottonseed Oil.” National Cottonseed Products Association, Inc. Memphis, Tenn.

56. Katavic V, Friesen W, Barton DL, Gossen KK, Giblin EM, Luciw T, An J, Zou Jitao, MacKenzie SL, Keller WA, Males D, Taylor DC (2001) “Improving erucic acid content in rapeseed through biotechnology.” Crop Sci. 41:739-747.

57. Katavic V, Reed DW, Taylor DC, Giblin EM, Barton DL, Zou J, MacKenzie SL, Covello PS, Kunst L (1995) “Alteration of seed fatty acid composition by an ethyl methanesulfonate-induced mutation in Arabidopsis thaliana affecting diacyglycerol acyltransferase activity.” Plant Physiol. 108:399-409.

58. Kaup MT, Froese CD, Thompson JE (2002) “A role for diacylglycerol acyltransferase during leaf senescence.” Plant Physiol. 129:1616-1612.

59. Ke J, Wen T, Nikolau B, Wurtele E (2000) “Coordinate regulation of the nuclear and plastidic genes coding for the subunits of the heteromeric acetyl-coenzyme A carboxylase.” Plant Physiol. 122:1057-1071.

60. Kerby TA, Keeley M, Watson M (1993) “Variation in fiber development as affected by source to sink relationships.” Proceedings: Beltwide Cotton Conf. Herber and Richter, eds. National Cotton Council. Memphis Tenn., pp. 1248-1251.

61. Kerby TA, Keely M, Johnson SR (1987) “Growth and development of Acala cotton, Bulletin 1921.” University of California, Division of Agriculture and Natural Resources, Oakland, Calif.

62. Kerby TA (1996) “Management considerations in cotton production.” Delta and Pine Land Company, Scott, Miss.

63. Kerby TA, Ruppenicker G (1992) “Canopy architecture and fiber quality variation by branch location.” Proceedings: Beltwide Cotton Production Research Conferences, p. 1069.

64. Kerby TA, Ruppenicker G (1989) “Node and Fruiting branch position effects on fiber and aeed quality characteristics.” Proceedings: Beltwide Cotton Production Research Conferences, pp. 98-100.

65. Khush GS (1999) “Strategies for increasing the yield potential of rice. Redesigning Rice Photosynthesis to Increase Yield.” Workshop Proceedings; Los Banos, Philippines; November 1999, Intl. Rice Res. Inst., pp 207-212.

66. Kimball BA (1983) “Carbon dioxide and agricultural yield: An assemblage and analysis of 430 prior observations.” Agron. J. 75:779-788.

67. Kridl JC, McCarter DW, Rose RE, Scherer DE, Knutzon DS, Radke SE, Knauf VC (1991) “Isolation and characterization of an expressed napin gene from Brassica rapa.” Seed Sci. Res. 1:209-219.

68. Kozaki A, Kamada K, Nagano Y, Iguchi H, Sasaki Y (2000) “Recombinant carboxyltransferase responsive to redox of pea plastidic acetyl-CoA carboxylase. J. Biol. Chem. 275:10702-10708.

69. Ku MSB, Agarie S, Nomura M, Fukayama H. Tsuchida H, Ono K, Hirose S, Toki S, Miyao M, Matsuoka M (1999) ” High-level expression of maize phosphoenolpyruvate carboxylase in transgenic rice plants.” Nat. Biotech. 17:76-80.

70. Kumar MVN, Kumar SS (2000) “Studies on character association and path coefficients for grain yield and oil content in maize (Zea mays L.).” Ann. Agric. Res. 21:73-78.

71. Landivar JA, Baker DN, Jenkins JN (1983) “Application of GOSSYM to genetic feasibility studies. I. Analysis of increasing photosynthesis, specific leaf weight and longevity of leaves in cotton.” Crop Sci. 23:504-510.

72. Last et al., (1991) Theor. Appl. Genet. 81:581-588.

73. Lawrence and Novak, (2001) Molecular Plant Breeding 8:139-146.

74. Lauterback B, Ganaway JR, Dever JK (2000) “Yield enhancement in cotton.” In: Genetic Control of Cotton Fiber and Seed Quality. Cotton Incorporated, Cary N.C., pp. 104-109.

75. Lepetit et al., (1992) Mol. Gen. Genet. 231:276-285.

76. Lewis H (2001) “A review of yield and quality trends and components in America upland cotton.” Proceedings: Beltwide Cotton Conferences. 2:1447-1453.

77. Liu Q, Singh SP, Green AG (2002a) “High-stearic and high-oleic cottonseed oils produced by hairpin RNA-mediated post-transcriptional gene silencing. Plant Physiol. 129:1732-1743.

78. Liu Q, Singh S, Green A (2002b) ” High-oleic and high-stearic cottonseed oils: nutritionally improved cooking oils developed using gene silencing.” J. Am. Coll. Nutr. 21:205S-211S.

79. Lu C, Hills M (2002) “Arabidopsis mutants deficient in diacylglycerol acyltransferase display increased sensitivity to abscisic acid, sugars, and osmotic stress during germination and seedling development.” Plant Physiol. 129:1352-1358.

80. Madoka Y, Tomizawa K-I, Mizoi J, Nishida I, Nagano Y, Sasaki Y (2002) “Chloroplast transformation with modified accD operon increases acetyl-CoA carboxylase and causes extension of leaf longevity and increase in seed yield in tobacco.” Plant Cell Physiol. 43:1518-1525.

81. Martinez A, Sparks C, Hart CA, Thompson J, Jepson 1 (1999) “Ecdysone agonist inducible transcription in transgenic tobacco plants.” Plant J. 19:97-106.

82. McElroy D, Zhang W, Cao J, Wu R (1990) “Isolation of an efficient actin promoter for use in rice transformation.” Plant Cell 2:163-171.

83. McD. Stewart J (1986) “Boll Development.” In Cotton Physiology. Stewart and Mauney, eds. The Cotton Foundation, Memphis Tenn., pp. 261-300.

84. Miyagawa Y, Tamoi M, Shigeoka S (2001) “Overexpression of a cyanobacterial fructose-1,6-/sedoheptulose-1,7-bisphosphatase in tobacco enhances photosynthesis and growth.” Nat. Biotech. 19:965-969.

85. Muskhelishvili G, Palm P, Zillig W (1993) “SSVL -encoded site-specific recombination system in Sulfolobus shibate.” Mol. Gen. Genet. 237:334-342.

86. Nykiforuk C, Laroche A, Wewelake RJ (1999) “Isolation and sequence analysis of a novel cDNA encoding a putative diacylglycerol acyltransferase from a microspore-derived cell suspension culture of Brassica napus L. cv Jet Neuf.” Plant Physiol. 120:99-123.

87. O'Hara P, Slabas AR, Fawcett T (2002) “Fatty acid and lipid biosynthetic genes are expressed at constant molar ratios but different absolute levels during embryogenesis.” Plant Physiol. 129:310-320.

88. Ohlorgge JB, Browse J (1995) “Lipid Biosynthesis.” Plant Cell 7:957-970.

89. Ohlrogge JB, Jaworski JG (1997) “Regulation of fatty acid synthesis. Annu. Rev. Plant Physiol. Mol. Biol. 48:109-136.

90. Oliver MJ, Velten J (2001) “Development of a genetically based seed technology protection system.” In: Dealing with Genetically Modified Crops. Wilson, Hou, and Hildebrand, eds. AOCS Press. Champaign Ill.

91. Ouwerkerk PB, Memelink J A (1999) “G-box element from the Catharanthus roseus strictosidine synthase (Str) gene promoter confers seed-specific expression in transgenic tobacco plants.” Mol. Gen. Genet. 261:635-643.

92. Pan G, Luetka K, Sadowski PD (1993) “Mechanism of cleavage and ligation by FLP recombinase: classification of mutations in FLP protein by in vitro complementation analysis.” Mol. Cell. Biol. 13:3167-3175.

93. Pellow J, Huang X, Anderson D, Meade T (2002) “Novel insect resistance traits from Dow AgroSciences.” Proceedings: Beltwide Cotton Conf. National Cotton Council, Memphis Tenn.

94. Pettigrew WT (2001) “Environmental effects on cotton fiber carbohydrate concentration and quality.” Crop Sci. 41:1108-1113.

95. Price GD, von Caemmerer S, Evans JR, Yu JW, Lloyd J, Oja V, Kell P, Harrison K, Gallagher A, Badger MR (1994) “Specific reduction of chloroplast carbonic anhydrase activity by antisense RNA in transgenic tobacco plants has a minor effect on photosynthetic C02 assimilation.” Planta 193:331-340.

96. Rask L, Ellerstrom M, Ezcurra I, Stalberg K, Wycliffe, P (1998) “Seed-specific regulation of the napin promoter in Brassica napus.” J. Plant Physiol. 152:595-599.

97. Rayburn ST, Gordon V, Britton RE (1989). Regional Cotton Variety Test, 1989.

98. Rayburn ST, Gordon V, Britton RE (1990). Regional Cotton Variety Test, 1990.

99. Rayburn ST, Keene ER (1997). National Cotton Variety Test, 1997.

100. Rayburn ST, Keene ER (1998). National Cotton Variety Test, 1998.

101. Rayburn ST, Keene ER (1999). National Cotton Variety Test, 1999.

102. Rayburn ST, Keene ER (2000). National Cotton Variety Test, 2000.

103. Rayburn ST, Keene ER (2001). National Cotton Variety Test, 2001.

104. Rayburn ST, Keene ER, Britton RE (1991). Regional Cotton Variety Test, 1991.

105. Rayburn ST, Keene ER, Britton RE (1992). National Cotton Variety Test, 1992.

106. Rayburn ST, Keene ER, Britton RE (1993). National Cotton Variety Test, 1993.

107. Rayburn ST, Keene ER, Britton RE (1994). National Cotton Variety Test, 1994.

108. Rayburn ST, Keene ER, Britton RE (1995). National Cotton Variety Test, 1995.

109. Rayburn ST, Keene ER, Britton RE (1996). National Cotton Variety Test, 1996.

110. Rayburn ST Jr, Keene ER (2001) 2001 National Cotton Variety Tests. http://msa.ars.usda.gov/stoneville/cgpr/ncvt/01 /2001book.htm.

111. Regierer B, Fernie AR, Springer F, Perez-Melis A, Leisse A, Koehl K, Willmitzer L, Geigenberger P, Kossmann J (2002) “Starch content and yield increase as a result of altering adenylate pools in transgenic plants.” Nat. Biotechnol. 20:1256-1257.

112. Reverdatto S, Beilinson V, Nielsen NC (1999) “A multisubunit acetyl coenzyme A carboxylase from soybean.” Plant Physiol. 119:961-978.

113. Richards RA (2000) “Selectable traits to increase crop photosynthesis and yield of grain crops.” J. Exp. Bot. 51:447-458.

114. Roesler K, Shintani D, Savage L, Boddupalli S, Ohlrogge J (1997) “Targeting of the Arabidopsis homomeric acetyl-coenzyme A carboxylase to plastids of rapeseeds.” Plant Physiol. 113 75-81.

115. Roesler K, Shorrosh B, Ohlrogge J (1994) “Structure and expression of an Arabidopsis Acetyl Coenzyme A Carboxylase gene.” Plant Physiol. 105:611-617.

116. Ruuska SA, Girke T, Benning C, Ohirogge JB (2002) “Contrapuntal networks of gene expression during Arabidopsis seed filling.” Plant Cell 14:1191-1206.

117. Sandoval JA, Huang ZH, Garrett DC, Gage DA, Chapman KD (1995) “N-acylphosphatidylethanolamine in dry and imbibing cottonseeds.” Plant Physiol. 109:269-275.

118. SAS (2002) JMP Version 5.0. SAS Institute Inc. Cary, N.C.

119. Sarmiento C, Ross JHE, Herman E, Murphy DJ (1997) “Expression and subcellular targeting of a soybean oleosin in transgenic rapeseed. Implications for the mechanism of oil-body formation in seed.” Plant J. 11:783-796.

120. Sasaki Y, Kosaki A, Ohmore A, Iguchi H, Nagano Y (2001) “Chloroplast RNA editing required for functional acetyl-CoA carboxyplase in plants.” J. Biol. Chem. 276:3937-3940.

121. Schena M, Lloyd AM, Davis RW (1991) “A steroid-inducible gene expression system for plant cells.” Proc. Natl. Acad. Sci. USA 88:10421-10425.

122. Sellwood C, Slabas AR, Rawsthorne S (2000) “Effects of manipulation expression of acetyl-CoA carboxylase I in Brassica napus L. embryos.” Biochem. Soc. Trans. 28:598-600.

123. Shanthi S, Nadarajan N, Backiyarani S (1999) “Genetic architecture of cotton seed oil content.” Madras Agric. J. 86:332-333.

124. Shen WH, Hohn B (1992) “Excision of a transposable element from a viral vector introduced into maize plants by agroinfection.” Plant J. 2:35-42.

125. Shintani DK, Roesler K, Shorrosh B, Savage L, Ohirogge JB (1997) “Antisense expression and overexpression of biotin carboxylase in tobacco leaves.” Plant Physiol. 114:881-886.

126. Shrestha R, Noordermeer MA, Van der Stelt M, Veldink GA, Chapman KD (2002) “N-acylethanolamines are metabolized by lipoxygenase and amidohydrolase in competing pathways during cottonseed imbibition.” Plant Physiol. 130:391-401.

127. Shorrosh BS, Savage LJ, Soll J, Ohlrogge JB (1996) “The pea chloroplast membrane-associated protein, IEP96, is a subunit of acetyl-CoA carboxylase.” Plant J. 10:261-268.

128. Smidansky ED, Clancy M, Meyer FD, Lanning SP, Blake NK, Talbert LE, Giroux MJ (2002) “Enhanced ADP-glucose pyrophosphorylase activity in wheat endosperm increases seed yield.” Proc. Natl. Acad. Sci. USA 99:1724-1729.

129. Smidansky ED, Martin JM, Hannah LC, Fisher AM, Giroux MJ (2002) “Seed yield and plant biomass increases in rice are conferred by deregulation of endosperm ADP-glucose pyrophosphorylase.” Planta Online December 2002.

130. Smith DL, Chareles TC, D'Aoust F, Driscoll BT, Prithiviraj B, Zhang H (2002) “Bradyrhizobium japonicum mutants allowing improved soybean yield in short season areas with cool spring soil temperatures.” Crop Sci. 42:1186-1190.

131. Somerville C, Browse J, Jaworski JG, Ohirogge JB (2000) “Lipids.” In Biochemistry and Molecular Biology of Plants. Buchanan, Gruissem and Jones, eds. Am. Soc. Plant Physiologist, Rockville Md.

132. Speed TR, Krief DR, Jividen G (1996) “Relationship between cotton seedling cold tolerance and physical and chemical properties.” Proceedings: Beltwide Cotton Conference V2: 1170-1171.

133. Stoutjeskijk P, Singh S, Liu Q, Hurlstone C, Waterhouse P, Green A (2002) “hpRNA A-mediated targeting of the Arabidopsis FAD2 gene gives highly efficient and stable silencing.” Plant Physiol. 129:1723-1731.

134. Suh MC, Schultz DJ, Ohlorogge JB (1999) “Isoforms of acyl carrier protein involved in seed-specific fatty acid synthesis.” Plant J. 17:679-688.

135. Sun J, Sakulringharoj C, Okita TW, Choi SB, Edwards GE, Ito H, Kato C, Matsui H (2001) “Increasing rice productivity and yield by manipulation of starch synthesis.” Novartis Foundation Symposium 236:135-152.

136. Sunilkumar G, Connell JP, Smith CW, Reddy AS, Rathore KS (2002) “Cotton α-globulin promoter: isolation and functional characteristics in transgenic cotton, Arabidopsis, and tobacco.” Transgenic Res. 11:347-359.

137. Sunilkumar G, Rathore KS (2001), “Transgenic Cotton: Factors Influencing Agrobacterium-mediated Transformation and Regeneration,” Mol. Breeding 8:37-52.

138. Taylor CB (1998) “Factories of the future? Metabolic engineering in plant cells. Plant Cell 10:641-644.

139. Taylor DC, Katavic V, Zou J, MacKenzie SL, Keller WA, An J, Friesen W, Varton DL, Pederson KK, Giblin EM, Ge Y, Dauk M, Sonntag C, Luciw T, Males D (2001) “Field testing of transgenic rapeseed cv. Hero transformed with a yeast sn-2 acyltransferase results in increased oil content, erucic acid content and seed yield.” Mol. Breeding 8:317-322.

140. Thelen JJ, Mekhedov S, Ohlrogge JB (2001) “Brassicaceae express multiple isoforms of biotin carboxyl carrier protein in a tissue-specific manner.” Plant Physiol. 125:2016-2028.

141. Townsend BJ, Llewellyn DJ (2002) “Spatial and temporal regulation of a soybean (Glycine max) lectin promoter in transgenic cotton (Gossypium hirsutum).” Funct. Plant Biol. 29:835-843.

142. Trealease RN, Miemyk JA, Choinski JS, Bortman SJ (1986) Synthesis and Compartmentation of Enzymes during Cottonseed Maturation.” In: Cotton Physiology. Chapter 29. Published by The Cotton Foundation.

143. Tzen JFC, Cao YZ, Laurent P, Ratnayake C, Huang AHC (1993) “Lipids, proteins, and structure of seed oil bodies from diverse species.” Plant Physiol. 101:267-276.

144. Unger E, Cigan AM, Trimnell M, Xu R-J, Kendall T, Roth B, Albersen M (2002) “A chimeric ecdysone receptor facilitates methoxyfenozide-dependent restoration of male fertility in ms45 maize.” Transgenic Res. 11:455465.

145. USDA-AMS (2002) Market News Reports-Cotton. United States Department of Agriculture, Agriculture Marketing Service. http://www.ams.usda.gov.

146. van Houdt H, Bleys A, Depicker A (2003) “RNA target sequences promote spreading of RNA silencing.” Plant Physiol. 131:245-253.

147. Van der Geest AH, Hall TC (1996) “A 68 bp element of the beta-phaseolin promoter functions as a seed-specific enhancer.” Plant Mol. Biol. 32:579-588.

148. Walker-Peach C, Velten J (1994) “Agrobacterium-mediated gene transfer to plant cells: cointegrate and binary vector systems.” In: Plant Molecular Biology Manual. Gelvin, Schilperoot and Verma, eds. Kluwer Publishing.

149. Wessler HG, Chapman KD, Benjamin RC (2002) “Genomic region from Gossypium hirsutum L. encoding a plastid targeted carbonic anhydrase isoform (CAH2).” NCBI AF482951.

150. White JA, Todd J, Newman T, Focks N, Girke T, Martinez de Ilarduya O, Jaworski JG, Ohirogge JB, Benning C (2000) “A new set of Arabidopsis expressed sequence tags from developing seeds. The metabolic pathway from carbohydrates to seed oil.” Plant Physiol. 124:1582-1594.

151. Wilmitzer L, Regierer B, Lloyd JR, Kossmann J, Geigenberger P, Femie A, Ritte G (2001) “Improving starch quality and yield in potato tubers.” Photosynthesis Res. 69:1-3.

152. Wullschleger SD, Oosterhuis DM (1990) “Photosynthetic Carbon Production and Use by Developing Cotton Leaves and Bolls.” Crop Sci. 30:1259-1264.

153. Yuan YL, Zhang TZ, Jing SR, Pan JJ, Zing CZ, Gou LP, Tang CM (2001) “Studies of the inheritance of seed qualities and the exploitation F2 heterosis in low Gossypol strains in upland cotton.” Acta Genetica Sinica 28:471-481.

154. Zhang H-X, Hodson JN, Williams JP, Blumwald E (2001) “Engineering salt-tolerant Brassica plants: Characterization of yield and seed oil quality in transgenic plants with increased vacuolar sodium accumulation.” Proc. Natl. Acad. Sci. USA 98:128.

155. Zou J, Brokx SJ, Taylor DC (1996) “Cloning of a cDNA encoding the 21.2k kDa oleosin isoform from Arabidopsis thaliana and a study of its expression In a mutant defective in diacylglycerol acyltransferase activity,” Plant Mol. Biol. 31:429-433.

156. Zou J, Katavic V, Giblin EM, Barton DL, MacKenzie SL, Keller WA, Hu X, Taylor DC (1997) “Modification of seed oil content and acyl composition in the Brassicaceae by expression of a yeast sn-2 acyltransferase gene.” Plant Cell 9:909-923.

157. Zou J, Niu Q-W, Chua N-H (2000) “An estrogen receptor-based transactivator XVE mediates highly inducible gene expression in transgenic plants.” Plant J. 24:265-273.

158. Zou J, Wei Y, Jako C, Kumar A, Selvaray G, Taylor DC (1999) “The Arabidopsis thaliana TAG1 mutant has a mutation in a diacylglycerol acyltransferase gene.” Plant J. 19:645-653.

Claims:

1. A reduced seed-oil content plant cell that expresses a seed-oil suppressing gene under control of a plant-active promoter, wherein said plant exhibits a reduction in seed-oil and a concomitant increase in plant carbohydrate, protein or both and wherein said seed-oil suppressing gene is selected from the group consisting of a mutant allele of a gene naturally occurring in said plant and a transgene.

2. A reduced seed-oil content plant cell of claim 1, which is selected from the group consisting of cotton, corn, soybean, canola and wheat.

3. A reduced seed-oil content plant cell of claim 2, which is a cotton plant cell.

4. A reduced seed-oil content plant which comprises a cell of claim 1.

5. A reduced seed-oil content plant of claim 4, wherein said cotton plant has enhanced fiber yield.

6. A reduced seed-oil content plant of claim 1, wherein said seed-oil suppressing gene is a mutant allele of a gene naturally occurring in said plant.

7. A reduced seed-oil content plant of claim 4, which is an elite cultivar.

8. A reduced seed-oil content plant of claim 4, which is a primitive cultivar.

9. A reduced seed-oil content plant of claim 4, wherein said seed-oil suppressing gene is introduced into the germplasm of said elite cultivar.

10. A reduced seed-oil content plant of claim 4, wherein said seed-oil suppressing gene controls seed-oil content by suppressing seed-oil biosynthesis.

11. A reduced seed-oil content plant of claim 4, wherein said seed-oil suppressing gene controls seed-oil content by suppressing seed-oil storage.

12. A reduced seed-oil content plant of claim 4, wherein said seed-oil suppressing gene is generated within the germplasm of said plant by random mutagenesis.

13. A reduced seed-oil content plant of claim 12, wherein said seed-oil suppressing gene is mutagenized by exposure to ethyl methanesulfonate.

14. A reduced seed-oil content plant of claim 4, wherein said seed-oil suppressing gene is identified and isolated from a mutagenized seed stock.

15. A reduced seed-oil content plant of claim 4, wherein expression of said seed-oil suppressing gene suppresses at least one biosynthetic step in oil biosynthesis.

16. A reduced seed-oil content plant of claim 4, wherein expression of said seed-oil suppressing gene suppresses a gene selected from the group consisting of carbonic anhydrase, ACCase, lysophosphatidic acid acyltransferase (LPAT), diacylglycerol acyltransferase (DGAT), oleosin and any combination thereof.

17. A reduced seed-oil content plant of claim 15, wherein said seed-oil suppressing gene suppresses a gene early in the oil biosynthetic pathway and a gene late in the oil biosynthetic pathway.

18. A reduced seed-oil content plant of claim 17, wherein said gene that is early in the seed-oil biosynthesis pathway is selected from the group consisting of the CA gene and the ACCase gene, and wherein said gene that is late in the seed-oil biosynthesis pathway is selected from the group consisting of the LPAT gene and the DGAT gene.

19. A reduced seed-oil content plant of claim 4, wherein said seed-oil suppressing gene is a transgene.

20. A reduced seed-oil content plant of claim 19, wherein expression of said transgene suppresses seed-oil biosynthesis.

21. A reduced seed-oil content plant of claim 19, wherein expression of said transgene suppresses at least one biosynthetic step in oil biosynthesis.

22. A reduced seed-oil content plant of claim 21, wherein expression of said transgene suppresses a gene selected from the group consisting of carbonic anhydrase, ACCase, lysophosphatidic acid acyltransferase, diacylglycerol acyltransferase, oleosin and any combination thereof.

23. A reduced seed-oil content plant of claim 21, wherein said transgene suppresses a gene early in the oil biosynthetic pathway and a gene late in the oil biosynthetic pathway.

24. A reduced seed-oil content plant of clam 21, wherein said seed-oil suppressing transgene is a nucleic acid that encodes an RNAi sequence for a gene that is early in the seed-oil biosynthesis pathway and for a gene that is late in the seed-oil biosynthesis pathway.

25. A reduced seed-oil content plant of claim 24, wherein said gene that is early in the seed-oil biosynthesis pathway is selected from the group consisting of the CA gene and the ACCase gene, and wherein said gene that is late in the seed-oil biosynthesis pathway is selected from the group consisting of the LPAT gene and the DGAT gene.

26. A reduced seed-oil content plant of claim 4, wherein said seed-oil suppressing gene is selected from the group consisting of a cosuppression directing nucleic acid, an antisense nucleic acid, a nucleic acid that encodes an immunomodulation protein, a nucleic acid that encodes a ribozyme, a nucleic acid that encodes a transcription factor suppressor and a nucleic acid that encodes an RNAi sequence.

27. A reduced seed-oil content plant of claim 19, wherein said transgene is operatively linked to a constitutive promoter.

28. A reduced seed-oil content plant of claim 27, wherein said constitutive promoter is selected from the group consisting of the 35S promoter from cauliflower mosaic virus, the maize ubiquitin promoter, the peanut chlorotic streak caulimovirus promoter, a Chlorella virus methyltransferase gene promoter, the full-length transcript promoter form figwort mosaic virus, the rice actin promoter, pEMU promoter, MAS promoter, the maize H3 histone promoter and an Agrobacterium gene promoter.

29. A reduced seed-oil content plant of claim 19, wherein said transgene is operatively linked to a seed-specific promoter.

30. A reduced seed-oil content plant of claim 29, wherein said seed-specific promoter is selected from the group consisting of the cotton alpha-globulin promoter, the napin gene promoter, the soybean alpha-conglycinin gene promoter, the soybean beta-conglycinin gene promoter and the soybean lectin promoter.

31. A reduced seed-oil content plant of claim 29, wherein said seed-specific promoter is generated by operable linkage of a genetic element that directs seed-specific expression to a core promoter sequence.

32. A reduced seed-oil content plant of claim 19, wherein said promoter is activated by application of an external stimulus.

33. A reduced seed-oil content plant of claim 32, wherein said seed-oil suppressing gene is expressed in the presence of said external stimulus.

34. A reduced seed-oil content plant of claim 32, wherein said external stimulus is copper, a benzenesulfonamide herbicide safener, a glucocorticosteroid hormone, estradiol and ecdysterodial activity.

35. A reduced seed-oil content plant of claim 32, wherein expression of said seed-oil suppressing gene, after activation, continues to be expressed in the absence of said external stimulus.

36. A reduced seed-oil content plant of claim 19, wherein said seed-oil suppressing gene is operably linked to a promoter selected from the group consisting of an inducible promoter and a repressible promoter.

37. A reduced seed-oil content plant of claim 36, wherein said inducible promoter is selected from the group consisting of the promoter from the ACE1 system, the promoter of the maize Intron 2 gene, the promoter of the Tet repressor from Tn10, the phosphate-deficiency responsive promoter from a phosphate-starvation responsive beta-glucosidase gene from Arabidopsis, the synthetic promoter containing a 235 bp sulfur deficiency response element from a soybean beta-conglycinin gene linked to a 35S core promoter sequence, the inducible promoter from a steroid hormone gene the transcriptional activity of which is induced by a glucocorticosteroid hormone and XVE.

38. A reduced seed-oil content plant of claim 36, wherein said promoter is a seed-specific promoter.

39. A reduced seed-oil content plant of claim 38, wherein said promoter is the cotton alpha-globulin promoter (AGP).

40. A reduced seed-oil content plant of claim 35, wherein said plant comprises an excisable blocking sequence that prevents expression of said seed-oil suppressing gene.

41. A reduced seed-oil content plant of claim 35, wherein the seed-oil content of said plant is reduced to a level of 1% to 17% of the fuzzy whole seed weight.

42. A reduced seed-oil content plant of claim 35, wherein stable pools of sucrose are generated in said plant that are available to increase, in a sustained fashion, the production of commercially valuable cellulosic, starch or protein macromolecules.

43. A reduced seed-oil content plant of claim 19, wherein said seed-oil suppressing transgene is a nucleic acid that encodes an RNAi sequence for a gene that is early in the seed-oil biosynthesis pathway and for a gene that is late in the seed-oil biosynthesis pathway.

44. A reduced seed-oil content plant of claim 43, wherein said gene that is early in the seed-oil biosynthesis pathway is selected from the group consisting of the CA gene and the ACCase gene, and wherein said gene that is late in the seed-oil biosynthesis pathway is selected from the group consisting of the LPAT gene and the DGAT gene.

45. A method for making a reduced seed-oil content plant of claim 4, which comprises:
(a) transfecting a plant cell with a transgene that suppresses a gene selected from the group consisting of carbonic anhydrase, ACCase, lysophosphatidic acid acyltransferase, diacylglycerol acyltransferase, oleosin and any combination thereof under control of a plant-active promoter; and
(b) regenerating a whole plant from said plant cell.

46. A method of claim 45, wherein said transgene suppresses a gene early in the oil biosynthetic pathway and a gene late in the oil biosynthetic pathway.

47. A method of claim 46, wherein said gene that is early in the seed-oil biosynthesis pathway is selected from the group consisting of the CA gene and the ACCase gene, and wherein said gene that is late in the seed-oil biosynthesis pathway is selected from the group consisting of the LPAT gene and the DGAT gene.

48. A method of claim 45, wherein said seed-oil suppressing transgene is a nucleic acid that encodes an RNAi sequence for a gene that is early in the seed-oil biosynthesis pathway and for a gene that is late in the seed-oil biosynthesis pathway.

49. A method of claim 45, wherein said seed-oil suppressing gene is selected from the group consisting of a cosuppression directing nucleic acid, an antisense nucleic acid, a nucleic acid that encodes an immunomodulation protein, a nucleic acid that encodes a ribozyme, a nucleic acid that encodes a transcription factor suppressor and a nucleic acid that encodes an RNAi sequence.

50. A method of claim 45, wherein said promoter is a constitutive promoter.

51. A method of claim 50, wherein said constitutive promoter is selected from the group consisting of the 35S promoter from cauliflower mosaic virus, the maize ubiquitin promoter, the peanut chlorotic streak caulimovirus promoter, a Chlorella virus methyltransferase gene promoter, the full-length transcript promoter form figwort mosaic virus, the rice actin promoter, pEMU promoter, MAS promoter, the maize H3 histone promoter and an Agrobacterium gene promoter.

52. A method of claim 45, wherein said promoter is a seed-specific promoter.

53. A method of claim 52, wherein said seed-specific promoter is selected from the group consisting of the cotton alpha-globulin promoter, the napin gene promoter, the soybean alpha-conglycinin gene promoter, the soybean beta-conglycinin gene promoter and the soybean lectin promoter.

54. A method of claim 52, wherein said seed-specific promoter is generated by operable linkage of a genetic element that directs seed-specific expression to a core promoter sequence.

55. A method of claim 52, wherein said promoter is activated by application of an external stimulus.

56. A method of claim 55, wherein said seed-oil suppressing gene is expressed in the presence of said external stimulus.

57. A method of claim 55, wherein said external stimulus is copper, a benzenesulfonamide herbicide safener, a glucocorticosteroid hormone, estradiol and ecdysterodial activity.

58. A method of claim 55, wherein expression of said seed-oil suppressing gene, after activation, continues to be expressed in the absence of said external stimulus.

59. A method of claim 55, wherein said promoter is selected from the group consisting of an inducible promoter and a repressible promoter.

60. A method of claim 57, wherein said inducible promoter is selected from the group consisting of the promoter from the ACE1 system, the promoter of the maize Intron 2 gene, the promoter of the Tet repressor from Tn10, the phosphate-deficiency responsive promoter from a phosphate-starvation responsive beta-glucosidae gene from Arabidopsis, the synthetic promoter containing a 235 bp sulfur deficiency response element from a soybean beta-conglycinin gene linked to a 35S core promoter sequence, the inducible promoter from a steroid hormone gene the transcriptional activity of which is induced by a glucocorticosteroid hormone and XVE.

61. A method of claim 59, wherein said promoter is a seed-specific promoter.

62. A method of claim 61, wherein said promoter is the cotton alpha-globulin promoter (AGP).

63. A method of claim 45, wherein the seed-oil content of said plant is reduced to a level of 1% to 17% or the fuzzy whole seed weight.

64. A method of claim 45, wherein stable pools of sucrose are generated in said plant that are available to increase, in a sustained fashion, the production of commercially valuable cellulosic, starch or protein macromolecules.

65. A breeding method for producing an enhanced yield self-pollinating plant that contains a yield enhancing gene, which comprises:
(a) providing an elite recurrent parent plant;
(b) providing a donor parent plant that contains said yield enhancing gene and that contains at least one phenotypic trait;
(c) crossing said elite recurrent parent plant with said donor parent plant to produce an F1 progeny plant;
(d) crossing said F1 progeny plant with said elite recurrent parent plant to produce a BC1F1 progeny plant that contains said yield enhancing gene;
(e) self-pollinating said BC1F1 progeny plant to produce a BC1F2 progeny plant that contains said yield enhancing gene;
(f) self-pollinating said BC1F2 progeny plant to produce BC1F2:3 plants that contain said yield enhancing gene;
(g) self-pollinating said BC1F2:3 plants;
(h) screening said BC1F2:3 plants for zygosity of said yield enhancing gene;
(i) collecting seed of said BC1F2:3 plants that are homozygous for said yield enhancing gene, which is BC1F2:4 seed; and
(j) germinating said seed to produce an enhanced yield self-pollinating plant that contains said yield enhancing gene.

66. A breeding method for producing an enhanced yield self-pollinating plant that contains a yield enhancing gene, which comprises:
(a) providing an elite recurrent parent plant;
(b) providing a donor parent plant that contains said yield enhancing gene and that contains at least one phenotypic trait;
(c) crossing said elite recurrent parent plant with said donor parent plant to produce an F1 progeny plant;
(d) crossing said F1 progeny plant with said elite recurrent parent plant to produce a BC1F1 progeny plant that contains said yield enhancing gene;
(e) self-pollinating said BC1F1 progeny plant to produce a BC1F2 progeny plant that contains said yield enhancing gene;
(f) self-pollinating said BC1F2 progeny plant one or more additional times to produce a BC1F3, BC1F4, BC1F5, BC1F6 or later generation of progeny plants that contain said yield enhancing gene;
(g) self-pollinating said progeny plants of step (f);
(h) screening said progency plants of step (f) for zygosity of said yield enhancing gene;
(i) collecting the seed of said progeny plants that are homozygous for said yield enhancing gene; and
(j) germinating said seed to produce an enhanced yield self-pollinating plant that contains said yield enhancing gene.

67. A breeding method of claim 65 or 66, wherein said screening for zygosity is performed by planting a progeny row and determining the extent to which plants in said progeny row exhibit a phenotypic trait characteristic of said yield enhancing gene.

68. A breeding method of claim 65 or 66, wherein said screening for zygosity is performed by testing for the expression of said yield enhancing gene.

69. A breeding method of claim 65 or 66, wherein said screening for zygosity is performed by testing for the presence of the gene in tissues of said progeny plants by PCR.

70. A breeding method for producing an enhanced yield self-pollinating plant that contains a yield enhancing gene, which comprises:
(a) providing an elite recurrent parent plant;
(b) providing a donor parent plant that contains said yield enhancing gene and that contains at least one phenotypic trait;
(c) crossing said elite recurrent parent plant with said donor parent plant to produce an F1 progeny plant;
(d) crossing said F1 progeny plant with said elite recurrent parent plant to produce a BC1F1 progeny plant that contains said yield enhancing gene;
(e) self-pollinating said BC1F1 progeny plant to produce a BC1F2 progeny plant that contains said yield enhancing gene;
(f) self-pollinating said BC1F2 progeny plant to produce BC1F2:3 plants that contain said yield enhancing gene;
(g) self-pollinating said BC1F2:3 plants;
(h) screening said BC1F2:3 plants for zygosity of said yield enhancing gene;
(i) collecting seed of said BC1F2:3 plants that contain said yield enhancing gene, which is BC1F3:4 seed;
(j) germinating said BC1F3:4 seed to produce BC1F3:4 plants;
(k) self-pollinating said BC1F3:4 plants;
(l) screening said BC1F3:4 plants for zygosity of said yield enhancing gene;
(m) collecting seed of said BC1F3:4 plants that are homozygous for said yield enhancing gene, which is BC1F3:5 seed; and
(n) germinating said seed to produce an enhanced yield self-pollinating plant that contains said yield enhancing gene.

71. A breeding method for producing an enhanced yield self-pollinating plant that contains a yield enhancing gene, which comprises:
(a) providing an elite recurrent parent plant;
(b) providing a donor parent plant that contains said yield enhancing gene and that contains at least one phenotypic trait;
(c) crossing said elite recurrent parent plant with said donor parent plant to produce an F1 progeny plant;
(d) crossing said F1 progeny plant with said elite recurrent parent plant to produce a BC1F1 progeny plant that contains said yield enhancing gene;
(e) self-pollinating said BC1F1 progeny plant to produce a BC1F2 progeny plant that contains said yield enhancing gene;
(f) self-pollinating said BC1F2 progeny plant one or more additional times to produce a BC1F3, BC1F4, BC1F5, BC1F6 or later generation of progeny plants that contain said yield enhancing gene;
(g) self-pollinating said progeny plants of step (f);
(h) screening said progeny plants of step (f) for zygosity of said yield enhancing gene;
(i) collecting seed of said progeny plants of step (f) that contain said yield enhancing gene;
(j) germinating said seed of step (i) to produce plants;
(k) self-pollinating said plants of step (j);
(l) screening said plants of step (j) for zygosity of said yield enhancing gene;
(m) collecting seed of said plants of step (j) that are homozygous for said yield enhancing gene; and
(n) germinating said seed to produce an enhanced yield self-pollinating plant that contains said yield-enhancing gene.

72. A breeding method of claim 70 or 71, wherein said screening for zygosity is performed by planting a progeny row and determining the extent to which plants in said progeny row exhibit a phenotypic trait characteristic of said yield enhancing gene.

73. A breeding method of claim 70 or 71, wherein said screening for zygosity is performed by testing for the expression of said yield enhancing gene.

74. A breeding method of claim 70 or 71, wherein said screening for zygosity is performed by testing for the presence of the gene in tissues of said progeny plants by PCR.

75. A breeding method for producing an enhanced yield self-pollinating plant that contains a yield enhancing gene, which comprises:
(a) providing an elite recurrent parent plant;
(b) providing a donor parent plant that contains said yield enhancing gene and that contains at least one phenotypic trait;
(c) crossing said elite recurrent parent plant with said donor parent plant to produce an F1 progeny plant;
(d) crossing said F1 progeny plant with said elite recurrent parent plant to produce a BC1F1 progeny plant that contains said yield enhancing gene;
(e) self-pollinating said BC1F1 progeny plant to produce a BC1F2 progeny plant that contains said yield enhancing gene;
(f) self-pollinating said BC1F2 progeny plant to produce BC1F2:3 plants that contain said yield enhancing gene;
(g) self-pollinating said BC1F2:3 plants;
(h) screening said BC1F2:3 plants for zygosity of said yield enhancing gene;
(i) collecting seed of said BC1F2:3 plants that contain said yield enhancing gene, which is BC1F3:4 seed;
(j) germinating said BC1F3:4 seed to produce BC1F3:4 plants;
(k) self-pollinating said BC1F3:4 plants;
(l) screening said BC1F3:4 plants for zygosity of said yield enhancing gene;
(m) collecting seed of said BC1F3:4 plants that contain said yield enhancing gene, which is BC1F4:5 seed;
(n) germinating said BC1F4:5 seed to produce BC1F4:5 plants;
(o) self-pollinating said BC1F4:5 plants;
(p) screening said BC1F4:5 plants for zygosity of said yield enhancing gene;
(q) collecting seed of said BC1F4:5 plants that are homozygous for said yield enhancing gene, which is BC1F4:6 seed; and
(r) germinating said seed to produce an enhanced yield self-pollinating plant that contains said yield enhancing gene.

76. A breeding method for producing an enhanced yield self-pollinating plant that contains a yield enhancing gene, which comprises:
(a) providing an elite recurrent parent plant;
(b) providing a donor parent plant that contains said yield enhancing gene and that contains at least one phenotypic trait;
(c) crossing said elite recurrent parent plant with said donor parent plant to produce an F1 progeny plant;
(d) crossing said F1 progeny plant with said elite recurrent parent plant to produce a BC1F1 progeny plant that contains said yield enhancing gene;
(e) self-pollinating said BC1F1 progeny plant to produce a BC1F2 progeny plant that contains said yield enhancing gene;
(f) self-pollinating said BC1F2 progeny plant one or more additional times to produce a BC1F3, BC1F4, BC1F5, BC1F6 or later generation of progeny plants that contain said yield enhancing gene;
(g) self-pollinating said progeny plants of step (f);
(h) screening said progeny plants of step (f) for zygosity of said yield enhancing gene;
(i) collecting seed of said progeny plants of step (f) that contain said yield enhancing gene;
(j) germinating said seed to produce plants;
(k) self-pollinating said plants of step (j);
(l) screening said plants of step (j) for zygosity of said yield enhancing gene;
(m) collecting seed of said plants of step (j) that contain said yield enhancing gene;
(n) germinating said seed of step (m) to produce plants;
(o) self-pollinating said plants of step (n);
(p) screening said plants of step (n) for zygosity of said yield enhancing gene;
(q) collecting seed of said plants of step (n) that are homozygous for said yield enhancing gene;
(r) germinating said seed of step (q) to produce an enhanced yield self-pollinating plant that contains said yield enhancing gene.

77. A breeding method of claim 75 or 76, wherein said screening for zygosity is performed by planting a progeny row and determining the extent to which plants in said progeny row exhibit a phenotypic trait characteristic of said yield enhancing gene.

78. A breeding method of claim 75 or 76,, wherein said screening for zygosity is performed by testing for the expression of said yield enhancing gene.

79. A breeding method of claim 75 or 76, wherein said screening for zygosity is performed by testing for the presence of the gene in tissues of said progeny plants by PCR.

80. A method of claim 75 which further comprises repeating steps (k)-(r) for generations of heterozygous progeny plants subsequent to BC1F4:5.

81. A method of any of claims 65, 66, 70, 71, 75 or 76 wherein said yield enhancing trait is selected from the group consisting of seed-oil suppression, delayed leaf senescence, enhanced leaf photosynthesis, enhanced leaf production of sucrose, enhance leaf export of sucrose, enhanced translocation of sucrose in the plant vasculature, reduced plant respiratory losses, reduced plant photorespiratory losses, reduced carbohydrate use in non-fruit plant tissue, enhanced movement of sucrose into the desired plant organ or tissue, and any combination thereof.

82. A method of any of claims 65, 66, 70, 71, 75 or 76 wherein said phenotypic trait is selected from the group consisting of dwarfing, short stature, more determinate growth habit, precocious flowering, intense flowering, rapid fruit development, medium to large seeds, large bolls, high fruit retention, high lint percent, low micronaire, cluster fruiting, insect protection, and any combination thereof.

83. A method of any of claims 65, 66, 70, 71, 75 or 76 wherein said elite recurrent parent plant is selected for a quality selected from the group consisting of yield, adaptation, fiber quality, agronomic performance and transgenic traits.

84. A method of any of claims 65, 66, 70, 71, 75 or 76 wherein said yield enhancing gene is selected from the group consisting of a mutant allele of a gene naturally occurring in said plant and a transgene.

85. A method of any of claims 65, 66, 70, 71, 75 or 76 wherein said donor parent plant is produced by directly transforming a recurrent plant containing said at least one phenotypic trait with said yield enhancing gene.

86. A method of any of claims 65, 66, 70, 71, 75 or 76 wherein said donor parent plant is produced by crossing a yield enhancing gene donor plant with a recurrent plant containing said at least one phenotypic trait and selecting progeny plants that contain both said yield enhancing gene and said at least one phenotypic trait.

87. A method of any of claims 65, 66, 70, 71, 75 or 76 wherein said donor parent plant is produced by crossing and backcrossing a yield enhancing gene donor plant with a recurrent plant containing said at least one phenotypic trait and selecting progeny plants that contain both said yield enhancing gene and said at least one phenotypic trait.
